SEQUENCE LISTING (148 sequences)

SEQ ID NO: 1 - DNA, 1244 bp, Homo sapiens
cgattctcatgcctcagcccaacgttagattggtttttccgagcctgtgaactccatggggtcgccagataaacgcaatcccggtggaccaacgcccaggacagacgctacgtaccaaccctgtgtcaagccgcaggcagcggttccggactctgggacttaacctaacgtggcggggcgggacttccggcgggaggcggaggcggaggcgcaggcgctggctggcaggtgtcgctaaccggacggtggtcgccagggcgagaggcgggagccggagaggtgaggcaggacccgggctccactgccgcctctccgagctcttgtgacgcggacctcagtgccaggatggctcggggcgagcggcggcgccgcgcagtgccggcagagggagtgcggacagccgagagggcggctcggggaggccccgggcgacgggacggccggggcggcgggccgcgtagcacggctggaggagtggctctggccgtcgtggtcctgtctttggccctgggtatgtcggggcgctgggtgctggcgtggtaccgtgcgcggcgggcggtcacgctgcactccgcgcctcctgtgttgcctgccgactcctccagccccgccgtggccccggacctcttctggggaacctaccgccctcacgtctacttcggcatgaagacccgcagcccgaagcccctcctcaccggtaaccgggccccagggcgggcaggcaggcggactcctaatctgggcgccccagcttgaagcgagagcaagggtgggaaggaatggatcaggagagttatgaagagggatgacccggagacccggagggacaggtcccctgctctttgacttacgtggccattctacccccaggactgatgtgggcgcagcagggcaccaccccggggactcctaagctcaggcacacgtgtgagcagggggacggtgtgggtccctatggctgggagttccacgacggcctctccttcgggcgccaacacatccaggatggggccttaaggctcaccactgagttcgtcaagaggcctgggggtcagcacggaggggactggagctggagagtgactgtagagcctcaggtcagggcctcaggacacccttttctcagcccagaccccctccatccttcccttgctgccagcaaatgcagcatgaagtgggctctggctgcctggcttaaatcctgccttaaacaactgcctgtataaccatttctatttaccatc

SEQ ID NO: 2 - DNA, 33 bp, Artificial Sequence
  Description of Artificial Sequence Synthetic oligonucleotide
atgcctcagccctaaacaactgcctgtataacc

SEQ ID NO: 3 - DNA, 109 bp, Artificial Sequence
  Description of Artificial Sequence Synthetic polynucleotide
cccgggttcaaacgattctcatgcctcagcccaacgttagattggtttttccgtaaatcctgccttaaacaactgcctgtataaccatttctatttaccatctctaagc

SEQ ID NO: 4 - DNA, 76 bp, Artificial Sequence
  Description of Artificial Sequence Synthetic oligonucleotide
cccgggttcaaacgattctcatgcctcagccctaaacaactgcctgtataaccatttctatttaccatctctaagc

SEQ ID NO: 5 - DNA, 39 bp, Homo sapiens
atgtgggcgcagcagggcaccaccccggggactcctaag

SEQ ID NO: 6 - DNA, 39 bp, Artificial Sequence
  Description of Artificial Sequence Synthetic oligonucleotide
atgtgggcgcagcagggcacccggggactcctaagctca

SEQ ID NO: 7 - DNA, 39 bp, Artificial Sequence
  Description of Artificial Sequence Synthetic oligonucleotide
atgtggggggggactcctaagctcaggcacacgtgtgag

SEQ ID NO: 8 - DNA, 39 bp, Artificial Sequence
  Description of Artificial Sequence Synthetic oligonucleotide
atgtgggcgcagcagggcaccacgccccggggactccta

SEQ ID NO: 9 - DNA, 39 bp, Artificial Sequence
  Description of Artificial Sequence Synthetic oligonucleotide
atgtgggcgcagcagggcaccacgggaccagcccgggga

SEQ ID NO: 10 - DNA, 45 bp, Homo sapiens
gactgatgtgggcgcagcagggcaccaccccggggactcctaagc

SEQ ID NO: 11 - DNA, 41 bp, Artificial Sequence
  Description of Artificial Sequence Synthetic oligonucleotide
gactgatgtgggcgcagcagggcacccggggactcctaagc

SEQ ID NO: 12 - DNA, 26 bp, Artificial Sequence
  Description of Artificial Sequence Synthetic oligonucleotide
gactgatgtgggggggactcctaagc

SEQ ID NO: 13 - DNA, 47 bp, Artificial Sequence
  Description of Artificial Sequence Synthetic oligonucleotide
gactgatgtgggcgcagcagggcaccacgccccggggactcctaagc

SEQ ID NO: 14 - DNA, 53 bp, Artificial Sequence
  Description of Artificial Sequence Synthetic oligonucleotide
gactgatgtgggcgcagcagggcaccacgggaccagcccggggactcctaagc

SEQ ID NO: 15 - DNA, 36 bp, Artificial Sequence
  Description of Artificial Sequence Synthetic oligonucleotide
tagaaaataaccaatataacgtttggagggaacatt

SEQ ID NO: 16 - DNA, 36 bp, Artificial Sequence
  Description of Artificial Sequence Synthetic oligonucleotide
cacataagacaggcagttgtttcagagagagcaaga

SEQ ID NO: 17 - DNA, 37 bp, Artificial Sequence
  Description of Artificial Sequence Synthetic oligonucleotide
tagaaaataaccaaatataacgtttggagggaacatt

SEQ ID NO: 18 - DNA, 36 bp, Artificial Sequence
  Description of Artificial Sequence Synthetic oligonucleotide
ctttttaaaactcatctaaagttgggcagagttaag

SEQ ID NO: 19 - DNA, 36 bp, Artificial Sequence
  Description of Artificial Sequence Synthetic oligonucleotide
acaatattacaggcagttttttatggtagagagctc

SEQ ID NO: 20 - DNA, 36 bp, Artificial Sequence
  Description of Artificial Sequence Synthetic oligonucleotide
ttcatttaaaccaacctaaagttggcagggatccca

SEQ ID NO: 21 - DNA, 36 bp, Artificial Sequence
  Description of Artificial Sequence Synthetic oligonucleotide
gaactgagacaggcagttgtttagtggtggagcaac

SEQ ID NO: 22 - DNA, 36 bp, Artificial Sequence
  Description of Artificial Sequence Synthetic oligonucleotide
acaagtcaaatcaatctaacttttggatggaatatt

SEQ ID NO: 23 - DNA, 36 bp, Artificial Sequence
  Description of Artificial Sequence Synthetic oligonucleotide
ccccttttacaggcagctgttttaggaggggaagca

SEQ ID NO: 24 - DNA, 36 bp, Artificial Sequence
  Description of Artificial Sequence Synthetic oligonucleotide
aataaaaacagcaatccaacgttggacagaagcctg

SEQ ID NO: 25 - DNA, 36 bp, Artificial Sequence
  Description of Artificial Sequence Synthetic oligonucleotide
agccataaacaggcaattgttaaaagaggaaaatac

SEQ ID NO: 26 - DNA, 27 bp, Artificial Sequence
  Description of Artificial Sequence Synthetic oligonucleotide
gactgatgtggggggggactcctaagc

SEQ ID NO: 27 - DNA, 21 bp, Homo sapiens
aaaaaccaatctaacgttggg

SEQ ID NO: 28 - DNA, 21 bp, Homo sapiens
aataaccaatataacgtttgg

SEQ ID NO: 29 - DNA, 21 bp, Homo sapiens
taaaactcatctaaagttggg

SEQ ID NO: 30 - DNA, 21 bp, Homo sapiens
ttaaaccaacctaaagttggc

SEQ ID NO: 31 - DNA, 21 bp, Homo sapiens
tcaaatcaatctaacttttgg

SEQ ID NO: 32 - DNA, 21 bp, Homo sapiens
aaacagcaatccaacgttgga

SEQ ID NO: 33 - DNA, 21 bp, Homo sapiens
ataaaccaatgtaaagtttgg

SEQ ID NO: 34 - DNA, 21 bp, Homo sapiens
taaaataaatctaacgttttg

SEQ ID NO: 35 - DNA, 21 bp, Homo sapiens
ataaaccaatcttactttagg

SEQ ID NO: 36 - DNA, 21 bp, Homo sapiens
aaaaataaatctaaagttgga

SEQ ID NO: 37 - DNA, 21 bp, Homo sapiens
taaaatgaatctaactttgag

SEQ ID NO: 38 - DNA, 21 bp, Homo sapiens
taaaagcaatcttacgtttgt

SEQ ID NO: 39 - DNA, 21 bp, Homo sapiens
taataccaatttaacggaggg

SEQ ID NO: 40 - DNA, 21 bp, Homo sapiens
caaaatcaatctaatgtagga

SEQ ID NO: 41 - DNA, 21 bp, Homo sapiens
ataatccactctaactttggg

SEQ ID NO: 42 - DNA, 21 bp, Homo sapiens
taaaactaatataaagttgga

SEQ ID NO: 43 - DNA, 21 bp, Homo sapiens
gaaaacaaatataaggttgga

SEQ ID NO: 44 - DNA, 21 bp, Homo sapiens
attaaccagtctaaggttggg

SEQ ID NO: 45 - DNA, 21 bp, Homo sapiens
aaaaacctatgtaacgtttag

SEQ ID NO: 46 - DNA, 21 bp, Homo sapiens
gaaaacaaatctaaccttatg

SEQ ID NO: 47 - DNA, 21 bp, Homo sapiens
aaaaactaatttaacattagg

SEQ ID NO: 48 - DNA, 21 bp, Homo sapiens
aaaaatccatctaatggtggg

SEQ ID NO: 49 - DNA, 21 bp, Homo sapiens
aaataacaagctaactttggg

SEQ ID NO: 50 - DNA, 21 bp, Homo sapiens
tcaggccaatctaatgttggg

SEQ ID NO: 51 - DNA, 21 bp, Homo sapiens
taacatcaatctgaagttggg

SEQ ID NO: 52 - DNA, 21 bp, Homo sapiens
caagaccaatgtatcattggg

SEQ ID NO: 53 - DNA, 21 bp, Homo sapiens
aaatcccaagctaaagttggg

SEQ ID NO: 54 - DNA, 21 bp, Homo sapiens
caaaacccatctacccttgcg

SEQ ID NO: 55 - DNA, 21 bp, Homo sapiens
caaaaccaaaataacattgga

SEQ ID NO: 56 - DNA, 21 bp, Homo sapiens
cacaaccattataaagttggg

SEQ ID NO: 57 - DNA, 21 bp, Homo sapiens
gaaaacaaatctaaaatttgg

SEQ ID NO: 58 - DNA, 21 bp, Homo sapiens
aaaaacccatctgccgttgag

SEQ ID NO: 59 - DNA, 21 bp, Homo sapiens
gaaaatcaatctgatggtggg

SEQ ID NO: 60 - DNA, 21 bp, Homo sapiens
taaaaccaacctcaagttggt

SEQ ID NO: 61 - DNA, 21 bp, Homo sapiens
caaaaccaacctaacttgggt

SEQ ID NO: 62 - DNA, 21 bp, Homo sapiens
caacaccaaaccaaagttggg

SEQ ID NO: 63 - DNA, 21 bp, Homo sapiens
gaaatccaaggtaaccttggg

SEQ ID NO: 64 - DNA, 21 bp, Homo sapiens
taaaagcaatgtaatattggg

SEQ ID NO: 65 - DNA, 21 bp, Homo sapiens
aaaaaagaatttaactttggg

SEQ ID NO: 66 - DNA, 21 bp, Homo sapiens
aaaaagtaatataacattggg

SEQ ID NO: 67 - DNA, 21 bp, Homo sapiens
ctaaacttatctgacgttggg

SEQ ID NO: 68 - DNA, 21 bp, Homo sapiens
aaaaaccaaactaatgttctg

SEQ ID NO: 69 - DNA, 21 bp, Homo sapiens
aaaaagcagtctcactttggg

SEQ ID NO: 70 - DNA, 21 bp, Homo sapiens
agacagcaatcaaacgttggg

SEQ ID NO: 71 - DNA, 21 bp, Homo sapiens
aaaaaaccatgtaaggttggg

SEQ ID NO: 72 - DNA, 21 bp, Homo sapiens
caaaataaaactaatgttggg

SEQ ID NO: 73 - DNA, 21 bp, Homo sapiens
taaaaccaataaaaagttgag

SEQ ID NO: 74 - DNA, 21 bp, Homo sapiens
aaaaaccaatttacctttgag

SEQ ID NO: 75 - DNA, 21 bp, Homo sapiens
taaaacaaatacaaggttggg

SEQ ID NO: 76 - DNA, 21 bp, Homo sapiens
gaaaaccaatctcagtttgga

SEQ ID NO: 77 - DNA, 21 bp, Homo sapiens
tatacaggcagttgtttaagg

SEQ ID NO: 78 - DNA, 21 bp, Homo sapiens
aagacaggcagttgtttcaga

SEQ ID NO: 79 - DNA, 21 bp, Homo sapiens
attacaggcagttttttatgg

SEQ ID NO: 80 - DNA, 21 bp, Homo sapiens
gagacaggcagttgtttagtg

SEQ ID NO: 81 - DNA, 21 bp, Homo sapiens
tttacaggcagctgttttagg

SEQ ID NO: 82 - DNA, 21 bp, Homo sapiens
aattcaggcaggtgtttaatg

SEQ ID NO: 83 - DNA, 21 bp, Homo sapiens
gaaagaggcagttgttttagg

SEQ ID NO: 84 - DNA, 21 bp, Homo sapiens
aatacatgcatttgtttatgg

SEQ ID NO: 85 - DNA, 21 bp, Homo sapiens
gataaaggcagttgctgaagg

SEQ ID NO: 86 - DNA, 21 bp, Homo sapiens
catacaggaagttgattaatg

SEQ ID NO: 87 - DNA, 21 bp, Homo sapiens
catttaggcagttgcttaagg

SEQ ID NO: 88 - DNA, 21 bp, Homo sapiens
tatacaggcagctgctaaagg

SEQ ID NO: 89 - DNA, 21 bp, Homo sapiens
tatactgacaattgtttaagg

SEQ ID NO: 90 - DNA, 21 bp, Homo sapiens
gatacagggtgttttttaagg

SEQ ID NO: 91 - DNA, 21 bp, Homo sapiens
gttacaggcatttgttcaaga

SEQ ID NO: 92 - DNA, 21 bp, Homo sapiens
agtacaggcagttgattaacc

SEQ ID NO: 93 - DNA, 21 bp, Homo sapiens
cagacaagcagttgttaaatg

SEQ ID NO: 94 - DNA, 21 bp, Homo sapiens
aaaacaggcaattgtttgaga

SEQ ID NO: 95 - DNA, 21 bp, Homo sapiens
gctgcaggcagttgtatgagg

SEQ ID NO: 96 - DNA, 21 bp, Homo sapiens
attacaggcaattatttaaga

SEQ ID NO: 97 - DNA, 21 bp, Homo sapiens
aataaatgcagttgttcaaga

SEQ ID NO: 98 - DNA, 21 bp, Homo sapiens
catggaggcagatgtttaaga

SEQ ID NO: 99 - DNA, 21 bp, Homo sapiens
aatccgggcagttgtgtaaag

SEQ ID NO: 100 - DNA, 21 bp, Homo sapiens
aattctggcagtggtttaagc

SEQ ID NO: 101 - DNA, 21 bp, Homo sapiens
aatataggcatttgtttaaat

SEQ ID NO: 102 - DNA, 21 bp, Homo sapiens
taaacaggcaattgttaaaag

SEQ ID NO: 103 - DNA, 21 bp, Homo sapiens
aaaacaggcagctgttaaaag

SEQ ID NO: 104 - DNA, 21 bp, Homo sapiens
aagacaggcagttgtgtgagt

SEQ ID NO: 105 - DNA, 21 bp, Homo sapiens
gatagagacagttgtgtaaga

SEQ ID NO: 106 - DNA, 21 bp, Homo sapiens
caaacaggaagttatttaaag

SEQ ID NO: 107 - DNA, 21 bp, Homo sapiens
aagagaggcagttgtattagg

SEQ ID NO: 108 - DNA, 21 bp, Homo sapiens
aattcaggcaattgttttaag

SEQ ID NO: 109 - DNA, 21 bp, Homo sapiens
cattgagggagttgtttaagc

SEQ ID NO: 110 - DNA, 21 bp, Homo sapiens
tttagaggaagttgtttaggg

SEQ ID NO: 111 - DNA, 21 bp, Homo sapiens
cacacagacagttgcttaggg

SEQ ID NO: 112 - DNA, 21 bp, Homo sapiens
aataaaggcagttctttaaac

SEQ ID NO: 113 - DNA, 21 bp, Homo sapiens
gatatagccagttttttaagt

SEQ ID NO: 114 - DNA, 21 bp, Homo sapiens
cataaaggcatttgttgaaga

SEQ ID NO: 115 - DNA, 21 bp, Homo sapiens
gatatagggagttatttaagt

SEQ ID NO: 116 - DNA, 21 bp, Homo sapiens
aaggcaggcagttggtcaagg

SEQ ID NO: 117 - DNA, 21 bp, Homo sapiens
aatataggcagttctttgagc

SEQ ID NO: 118 - DNA, 21 bp, Homo sapiens
caaacacacagttgtttaaag

SEQ ID NO: 119 - DNA, 21 bp, Homo sapiens
tattcaggcagttgtaaaaga

SEQ ID NO: 120 - DNA, 21 bp, Homo sapiens
tatagaggcagttgcttatgt

SEQ ID NO: 121 - DNA, 21 bp, Homo sapiens
tatgctgggagttgtttaaag

SEQ ID NO: 122 - DNA, 21 bp, Homo sapiens
tgttcaggctgttgttcaagg

SEQ ID NO: 123 - DNA, 21 bp, Homo sapiens
tatactggctgttgcttaaga

SEQ ID NO: 124 - DNA, 21 bp, Homo sapiens
attacaggcagatctttgagg

SEQ ID NO: 125 - DNA, 21 bp, Homo sapiens
catggaggcaggtgttttagg

SEQ ID NO: 126 - DNA, 21 bp, Homo sapiens
tataaaagcaattgtttaatg

SEQ ID NO: 127 - DNA, 20 bp, Artificial Sequence
  Description of Artificial Sequence Synthetic oligonucleotide
aagccttcaggcagcatctt

SEQ ID NO: 128 - DNA, 20 bp, Artificial Sequence
  Description of Artificial Sequence Synthetic oligonucleotide
actggtcaccgaagtgttca

SEQ ID NO: 129 - DNA, 20 bp, Artificial Sequence
  Description of Artificial Sequence Synthetic oligonucleotide
gcccaactggataaggaccc

SEQ ID NO: 130 - DNA, 20 bp, Artificial Sequence
  Description of Artificial Sequence Synthetic oligonucleotide
gcatgtggcatcatcagacc

SEQ ID NO: 131 - DNA, 20 bp, Artificial Sequence
  Description of Artificial Sequence Synthetic oligonucleotide
ctgcttccctgcccacttct

SEQ ID NO: 132 - DNA, 20 bp, Artificial Sequence
  Description of Artificial Sequence Synthetic oligonucleotide
cgttctgtggcaccataagc

SEQ ID NO: 133 - DNA, 20 bp, Artificial Sequence
  Description of Artificial Sequence Synthetic oligonucleotide
agtacgcagagagctcccga

SEQ ID NO: 134 - DNA, 20 bp, Artificial Sequence
  Description of Artificial Sequence Synthetic oligonucleotide
cccaggtccatggaaaccaa

SEQ ID NO: 135 - DNA, 20 bp, Artificial Sequence
  Description of Artificial Sequence Synthetic oligonucleotide
cctccatccctcaggagtgc

SEQ ID NO: 136 - DNA, 20 bp, Artificial Sequence
  Description of Artificial Sequence Synthetic oligonucleotide
tgcgtgccccattcctagtc

SEQ ID NO: 137 - DNA, 20 bp, Artificial Sequence
  Description of Artificial Sequence Synthetic oligonucleotide
ctcgctcaccggggtatctg

SEQ ID NO: 138 - DNA, 20 bp, Artificial Sequence
  Description of Artificial Sequence Synthetic oligonucleotide
ctggcacaggtgtccagggt

SEQ ID NO: 139 - DNA, 20 bp, Artificial Sequence
  Description of Artificial Sequence Synthetic oligonucleotide
agggttgaccttggatgagc

SEQ ID NO: 140 - DNA, 20 bp, Artificial Sequence
  Description of Artificial Sequence Synthetic oligonucleotide
cccattagcccgggatcaca

SEQ ID NO: 141 - DNA, 20 bp, Artificial Sequence
  Description of Artificial Sequence Synthetic oligonucleotide
tttgagaagctggctggcga

SEQ ID NO: 142 - DNA, 20 bp, Artificial Sequence
  Description of Artificial Sequence Synthetic oligonucleotide
ctggctcctggaggggtaac

SEQ ID NO: 143 - DNA, 20 bp, Artificial Sequence
  Description of Artificial Sequence Synthetic oligonucleotide
cctgctaacctgcgttctgt

SEQ ID NO: 144 - DNA, 20 bp, Artificial Sequence
  Description of Artificial Sequence Synthetic oligonucleotide
ggcaggctttgcctcatttc

SEQ ID NO: 145 - DNA, 20 bp, Artificial Sequence
  Description of Artificial Sequence Synthetic oligonucleotide
gtgcaattactgccatgcct

SEQ ID NO: 146 - DNA, 20 bp, Artificial Sequence
  Description of Artificial Sequence Synthetic oligonucleotide
ccttcctgcctgttgccatt

SEQ ID NO: 147 - DNA, 2867 bp, Homo sapiens
gcaggcgctggctggcaggtgtcgctaaccggacggtggtcgccagggcgagaggcgggagccggagaggtgaggcaggacccgggctccactgccgcctctccgagctcttgtgacgcggacctcagtgccaggatggctcggggcgagcggcggcgccgcgcagtgccggcagagggagtgcggacagccgagagggcggctcggggaggccccgggcgacgggacggccggggcggcgggccgcgtagcacggctggaggagtggctctggccgtcgtggtcctgtctttggccctgggtatgtcggggcgctgggtgctggcgtggtaccgtgcgcggcgggcggtcacgctgcactccgcgcctcctgtgttgcctgccgactcctccagccccgccgtggccccggacctcttctggggaacctaccgccctcacgtctacttcggcatgaagacccgcagcccgaagcccctcctcaccggactgatgtgggcgcagcagggcaccaccccggggactcctaagctcaggcacacgtgtgagcagggggacggtgtgggtccctatggctgggagttccacgacggcctctccttcgggcgccaacacatccaggatggggccttaaggctcaccactgagttcgtcaagaggcctgggggtcagcacggaggggactggagctggagagtgactgtagagcctcaggactcaggtacttctgccctccctttggtctccctgttcttctatgtggtgacagatggcaaggaagtcctactaccagaggttggggccaaggggcagttgaagtttatcagtgggcacaccagtgaacttggtgacttccgctttacacttttgccaccaaccagtccaggggatacagcccccaagtatggcagctacaatgtcttctggacctccaacccaggactgcccctgctgacagagatggtaaagagtcgcctaaatagctggtttcagcatcggcccccaggggccccccctgaacgctacctcggcttgccaggatccctgaagtgggaggacagaggtccaagtgggcaagggcaggggcagttcttgatacagcaggtgaccctgaaaattcccatttccatagagtttgtgtttgaatcaggcagtgcccaggcaggaggaaatcaagccctgccaagactggcaggcagtctactgacccaggccctggagagccatgctgaaggctttagagagcgctttgagaagaccttccagctgaaggagaagggcctgagctctggcgagcaggttttgggtcaggctgccctcagcggcctccttggtggaattggctacttctacggacaagggctggtattgccagacatcggggtggaagggtctgagcagaaggtggacccagccctctttccacccgtacctctttttacagcagtgccctcccggtcattcttcccacgaggcttcctttgggatgaaggctttcaccagctggtggttcagcggtgggatccctccctcacccgggaagcccttggccactggctggggctgctaaatgctgatggctggattgggagggagcagatactgggggatgaggcccgagcccgggtgcctccagaattcctagtacaacgagcagtccacgccaaccccccaaccctacttttgcctgtagcccatatgctagaggttggtgaccctgacgacttggctttcctccgaaaggccttgccccgcctgcatgcctggttttcctggctccatcagagccaggcaggcccactgccactatcttaccgctggcggggacgggaccctgccttaccaaccttactgaaccccaagaccctaccctctgggctggatgactacccccgggcttcacacccttcagtaaccgagcggcacctggacctgcgatgttgggtggcactgggtgcccgtgtgctgacgcggctggcagagcatctgggtgaggctgaggtagctgctgagctgggcccactggctgcctcactggaggcagcagagagcctggatgagctgcactgggccccagagctaggagtctttgcagactttgggaaccacacaaaagcagtacagctgaagcccaggccccctcaggggctcgttcgggtggtgggtcggccccaacctcaactgcagtatgtagatgctcttggctatgtcagtctttttcccttgctgctgcgactgctggaccccacctcatcccgccttgggcccctgctggacattctagccgacagccgccatctctggagcccctttggtttacgctcccttgcagcctccagctccttttatggccagcgcaattcagagcatgatcccccctactggcggggtgctgtgtggctcaatgtcaactacctggctttgggagcactccaccactatgggcatctggagggtcctcaccaggctcgggctgccaaactccacggtgagctccgtgccaacgtggtaggcaatgtatggcgccagtaccaggctacaggctttctttgggagcagtacagtgaccgcgatgggcgaggcatgggctgccgccctttccacggctggaccagccttgtcttactggccatggctgaagactactgaagggagggagaggaggggagccaagacactcatgccactctggctctgaagggacaaaggcttctggcttttgcccccagccccttggataccagtaattcaaaccttcctcatttcatctcaggtgtctccttgctgtcatcccacatagccctggggtgaatgtgaatccagagtctatttttctaaataaattggaaaaaacattttgaactcta

SEQ ID NO: 148 - protein, 837 aa, Homo sapiens
MARGERRRRAVPAEGVRTAERAARGGPGRRDGRGGGPRSTAGGVALAVVVLSLALGMSGRWVLAWYRARRAVTLHSAPPVLPADSSSPAVAPDLFWGTYRPHVYFGMKTRSPKPLLTGLMWAQQGTTPGTPKLRHTCEQGDGVGPYGWEFHDGLSFGRQHIQDGALRLTTEFVKRPGGQHGGDWSWRVTVEPQDSGTSALPLVSLFFYVVTDGKEVLLPEVGAKGQLKFISGHTSELGDFRFTLLPPTSPGDTAPKYGSYNVFWTSNPGLPLLTEMVKSRLNSWFQHRPPGAPPERYLGLPGSLKWEDRGPSGQGQGQFLIQQVTLKIPISIEFVFESGSAQAGGNQALPRLAGSLLTQALESHAEGFRERFEKTFQLKEKGLSSGEQVLGQAALSGLLGGIGYFYGQGLVLPDIGVEGSEQKVDPALFPPVPLFTAVPSRSFFPRGFLWDEGFHQLVVQRWDPSLTREALGHWLGLLNADGWIGREQILGDEARARVPPEFLVQRAVHANPPTLLLPVAHMLEVGDPDDLAFLRKALPRLHAWFSWLHQSQAGPLPLSYRWRGRDPALPTLLNPKTLPSGLDDYPRASHPSVTERHLDLRCWVALGARVLTRLAEHLGEAEVAAELGPLAASLEAAESLDELHWAPELGVFADFGNHTKAVQLKPRPPQGLVRVVGRPQPQLQYVDALGYVSLFPLLLRLLDPTSSRLGPLLDILADSRHLWSPFGLRSLAASSSFYGQRNSEHDPPYWRGAVWLNVNYLALGALHHYGHLEGPHQARAAKLHGELRANVVGNVWRQYQATGFLWEQYSDRDGRGMGCRPFHGWTSLVLLAMAEDY

(19) United States
(12) Patent Application Publication
(10) Pub. No.: US 2024/0336939 A1
(43) Pub. Date: Oct. 10, 2024

(54) CRISPR INDUCED DISRUPTION OF MOGS GENE

(71) Applicant: TEMPLE UNIVERSITY - OF THE COMMONWEALTH SYSTEM OF HIGHER EDUCATION, Philadelphia, PA (US)

(72) Inventors: Kamel KHALILI, Bala Cynwyd, PA (US); Rafal KAMINSKI, Philadelphia, PA (US)

(21) Appl. No.: 18/566,446

(22) PCT Filed: Jun. 2, 2022
(86) PCT No.: PCT/US22/31954

§ 371 Date: Dec. 1, 2023

Related U.S. Application Data
(60) Provisional application No. 63/305,081, filed on Jan. 31, 2022.
(60) Provisional application No. 63/196,042, filed on Jun. 2, 2021.

(51) Int. Cl. C12N 15/90; C12N 9/22; C12N 15/11

(52) U.S. Cl. CPC C12N 15/907; C12N 9/22; C12N 15/11

(57) ABSTRACT

Compositions include CRISPR-associated endonuclease, and one or more isolated nucleic acid sequences encoding gRNAs, wherein each gRNA is complementary to a target sequence in a retroviral genome. At least one endonuclease targets a Mannosyl Oligosaccharide Glucosidase (MOGS).

### CROSS REFERENCE TO RELATED APPLICATIONS

This Application claims the benefit of U.S. Provisional Application 63/305,081 filed on Jan. 31, 2022 and U.S. Provisional Application 63/196,042 filed on Jun. 2, 2021. The entire contents of these applications are incorporated herein by reference in their entirety.

### STATEMENT REGARDING FEDERALLY SPONSORED RESEARCH

This invention was made with U.S. government support under grant number R01MH115860 awarded by the National Institutes of Health. The U.S. government may have certain rights in the invention.

### SEQUENCE LISTING

The instant application contains a Sequence Listing which has been submitted electronically in ASCII format and is hereby incorporated by reference in its entirety. Said ASCII copy, created on May 23, 2024, is named 348382_01201_SL.txt and is 37,993 bytes in size.

### FIELD

The present disclosure relates to compositions and methods that target a retroviral genome and a viral receptor, for example human immunodeficiency virus (HIV). The compositions, which can include nucleic acids encoding a Clustered Regularly Interspace Short Palindromic Repeat (CRISPR) associated endonuclease and a guide RNA sequence complementary to a target sequence in a human immunodeficiency virus and/or a viral receptor can be administered to a subject having or at risk for contracting an HIV infection.

### BACKGROUND

The envelope protein of HIV-1 is heavily glycosylated with a total of 24 N-linked glycosylation sites covering much of its surface. It has been shown that these glycosylations are essential for the proper intracellular proteolytic maturation of the envelope precursor glycoprotein gp160 into gp41 and gp120 and for post-CD4 binding functions of mature gp120. Inhibition of mannosyl-oligosaccharide glucosidase (MOGS, α-glucosidase 1), a key enzyme in processing N-linked glycan, leads to incomplete processing of viral envelope resulting in a reduction of virion infectivity and cell-to-cell spread of virus infection by suppression of cell fusion events.

### SUMMARY

The present disclosure provides compositions and methods relating to treatment and prevention of retroviral infections, for example, the human immunodeficiency virus HIV-1. The compositions and methods target the retroviral genome, a viral receptor or combinations thereof.

Specifically, the present disclosure provides compositions including a nucleic acid sequence encoding a CRISPR-associated endonuclease, and one or more isolated nucleic acid sequences encoding gRNAs, wherein each gRNA is complementary to a target sequence in a retroviral genome. In a preferred embodiment, two or more gRNAs are included in the composition, with each gRNA directing a Cas endonuclease to a different target site in integrated retroviral DNA. In some embodiments, at least one endonuclease targets a Mannosyl Oligosaccharide Glucosidase (MOGS). In another embodiment, a composition comprises two of more endonucleases targeted to a retroviral genome and two or more endonucleases targeted to MOGS.

In certain embodiments, a composition for preventing or treating a retroviral infection in vitro or in vivo, comprises: a first isolated nucleic acid sequence encoding a first Clustered Regularly Interspaced Short Palindromic Repeat (CRISPR)-associated endonuclease and at least one guide RNA (gRNA), the gRNA being complementary to a target sequence in the integrated retroviral DNA; a second isolated nucleic acid sequence encoding a second Clustered Regularly Interspaced Short Palindromic Repeat (CRISPR)-associated endonuclease and at least one guide RNA (gRNA), the gRNA being complementary to a target sequence in the integrated retroviral DNA; a third isolated nucleic acid sequences encoding a third Clustered Regularly Interspaced Short Palindromic Repeat (CRISPR)-associated endonuclease and at least one guide RNA (gRNA), the gRNA being complementary to a target sequence in a mannosyl-oligosaccharide glucosidase (MOGS) gene; a fourth isolated nucleic acid sequences encoding a fourth Clustered Regularly Interspaced Short Palindromic Repeat (CRISPR)-associated endonuclease and at least one guide RNA (gRNA), the gRNA being complementary to a target sequence in a mannosyl-oligosaccharide glucosidase (MOGS) gene regulatory region. In certain embodiments, the first and second target sequences comprise one or more nucleic acid sequences in the retrovirus, the target sequences comprising: long terminal repeat (LTR) nucleic acid sequences, Gag nucleic acid sequences, nucleic acid sequences encoding structural proteins, non-structural proteins or combinations thereof. In certain embodiments, intervening sequences between the first and second target sequences are excised. In certain embodiments, the target sequences comprise one or more intron sequences, exon sequences and combinations thereof of the MOGS gene. In certain embodiments, the regulatory target sequences of the MOGS gene comprise a promoter sequence, an enhancer, coding or non-coding regions associated with the MOGS gene. In certain embodiments, intervening sequences between the first and second target sequences are excised. In certain embodiments, the retrovirus is a human immunodeficiency virus (HIV).

In certain embodiments, a composition for preventing or treating a human immunodeficiency virus (HIV) infection, comprises at least two isolated nucleic acid sequences wherein: a first isolated nucleic acid sequence encodes a first Clustered Regularly Interspaced Short Palindromic Repeat (CRISPR)-associated endonuclease and at least one guide RNA (gRNA), the gRNA being complementary to a target sequence in encoding a third Clustered Regularly Interspaced Short Palindromic Repeat (CRISPR)-associated endonuclease and at least one guide RNA (gRNA), the gRNA being complementary to a target sequence in a mannosyl-oligosaccharide glucosidase (MOGS) gene; a second isolated nucleic acid sequences encoding a fourth Clustered Regularly Interspaced Short Palindromic Repeat (CRISPR)-associated endonuclease and at least one guide RNA (gRNA), the gRNA being complementary to a target sequence in a mannosyl-oligosaccharide glucosidase (MOGS) gene. In certain embodiments, the target sequences of the MOGS gene comprise promoter sequences, enhancer sequences, intron sequences, exon sequences and combinations thereof. and combinations thereof. In certain embodiments, the composition further comprises two or more isolated nucleic acid sequences encoding two or more (CRISPR)-associated endonucleases and guide RNAs (gRNAs) having complementarity to two or more target sequences, the target sequences comprising HIV sequences, sequences in receptors used by HIV for attachment and/or infection of a cell, and combinations thereof. In certain embodiments, the at least one receptor comprises CCR5, CD4, variants or combinations thereof. In certain embodiments, the HIV target sequences comprise one or more nucleic acid sequences comprising: long terminal repeat (LTR) nucleic acid sequences, Gag nucleic acid sequences, nucleic acid sequences encoding structural proteins, non-structural proteins or combinations thereof. In certain embodiments, the composition further comprises administering a therapeutically effective amount of at least one antiretroviral agent.

In certain embodiments, a method of preventing and treating infection by a retrovirus in vitro or in vivo, comprises administering at least two isolated nucleic acid sequences wherein: a first isolated nucleic acid sequence encodes a first Clustered Regularly Interspaced Short Palindromic Repeat (CRISPR)-associated endonuclease and at least one guide RNA (gRNA), the gRNA being complementary to a target sequence in encoding a third Clustered Regularly Interspaced Short Palindromic Repeat (CRISPR)-associated endonuclease and at least one guide RNA (gRNA), the gRNA being complementary to a target sequence in a mannosyl-oligosaccharide glucosidase (MOGS) gene; a second isolated nucleic acid sequences encoding a second Clustered Regularly Interspaced Short Palindromic Repeat (CRISPR)-associated endonuclease and at least one guide RNA (gRNA), the gRNA being complementary to a target sequence in a mannosyl-oligosaccharide glucosidase (MOGS) gene; wherein intervening sequences between the first and second target sequences are excised. In certain embodiments, the target sequences of the MOGS gene comprise promoter sequences, enhancer sequences, intron sequences, exon sequences and combinations thereof. In certain embodiments, the method further comprises administering two or more isolated nucleic acid sequences encoding two or more (CRISPR)-associated endonucleases and guide RNAs (gRNAs) having complementarity to two or more target sequences, the target sequences comprising HIV sequences, sequences in receptors used by HIV for attachment and/or infection of a cell, and combinations thereof. In certain embodiments, the at least one receptor comprises CCR5, CD4, variants or combinations thereof. In certain embodiments, the HIV target sequences comprise one or more nucleic acid sequences comprising: long terminal repeat (LTR) nucleic acid sequences, Gag nucleic acid sequences, nucleic acid sequences encoding structural proteins, non-structural proteins or combinations thereof. In certain embodiments, the method further comprises administering a therapeutically effective amount of at least one antiretroviral agent.

In certain embodiments, the CRISPR-associated endonuclease is a Type I, Type II, or Type III Cas endonuclease. In certain embodiments, the CRISPR-associated endonuclease is a Cas9 endonuclease, a Cas12 endonuclease, a Cas 13 endonuclease, a CasX endonuclease, a Caso endonuclease or variants thereof. In certain embodiments, the CRISPR-associated endonuclease is a Cas9 nuclease or variants thereof. In certain embodiments, the Cas9 nuclease is a Staphylococcus aureus Cas9 nuclease. In certain embodiments, the Cas9 variant comprises one or more point mutations, relative to wildtype Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In certain embodiments, a Cas9 variant comprises a human-optimized Cas9; a nickase mutant Cas9; saCas9; enhanced-fidelity SaCas9 (efSaCas9); SpCas9 (K855a); SpCas9 (K810A/K1003A/r1060A); SpCas9 (K848A/K1003A/R1060A); SpCas9 N497A, R661A, Q695A, Q926A; SpCas9 N497A, R661A, Q695A, Q926A, D1135E; SpCas9 N497A, R661A, Q695A, Q926A L169A; SpCas9 N497A, R661A, Q695A, Q926A Y450A; SpCas9 N497A, R661A, Q695A, Q926A M495A; SpCas9 N497A, R661A, Q695A, Q926A M694A; SpCas9 N497A, R661A, Q695A, Q926A H698A; SpCas9 N497A, R661A, Q695A, Q926A, D1135E, L169A; SpCas9 N497A, R661A, Q695A, Q926A, D1135E, Y450A; SpCas9 N497A, R661A, Q695A, Q926A, D1135E, M495A; SpCas9 N497A, R661A, Q695A, Q926A, D1135E, M694A; SpCas9 N497A, R661A, Q695A, Q926A, D1135E, M698A; SpCas9 R661A, Q695A, Q926A; SpCas9 R661A, Q695A, Q926A, D1135E; SpCas9 R661A, Q695A, Q926A, L169A; SpCas9 R661A, Q695A, Q926A Y450A; SpCas9 R661A, Q695A, Q926A M495A; SpCas9 R661A, Q695A, Q926A M694A; SpCas9 R661A, Q695A, Q926A H698A; SpCas9 R661A, Q695A, Q926A D1135E L169A; SpCas9 R661A, Q695A, Q926A D1135E Y450A; SpCas9 R661A, Q695A, Q926A D1135E M495A; or SpCas9 R661A, Q695A, Q926A, D1135E or M694A. In certain embodiments, the CRISPR-associated endonuclease is optimized for expression in a human cell.

In certain embodiments, the compositions are administered in a pharmaceutical composition comprising one or more vectors encoding one or more of the isolated nucleic acid sequences embodied herein.

In certain embodiments, the isolated nucleic acid sequences are included in at least one expression vector comprising: a lentiviral vector, an adenovirus vector, an adeno-associated virus vector, a vesicular stomatitis virus (VSV) vector, a pox virus vector, and a retroviral vector. In certain embodiments, the expression vector comprises: a lentiviral vector, an adenoviral vector, or an adeno-associated virus vector. In certain embodiments, the adeno-associated virus (AAV) vector is AV1, AAV2, AAV3, AAV4, AAV5, AAV6, AAV7, AAV8, AAV9, AAV10, AAV11, AAVDJ, or AAVDJ/8. In certain embodiments, the vector comprising the nucleic acid further comprises a promoter. In certain embodiments, the promoter comprises a ubiquitous promoter, a tissue-specific promoter, an inducible promoter or a constitutive promoter.

Any compositions or methods provided herein can be combined with one or more of any of the other compositions and methods provided herein.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “anti-viral agent” as used herein, refers to any molecule that is used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof. The term also refers to non-nucleoside reverse transcriptase inhibitors (NNRTIs), nucleoside reverse transcriptase inhibitors (NRTIs), analogs, variants etc.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “eradication” of a retrovirus, e.g. human immunodeficiency virus (HIV), as used herein, means that that virus is unable to replicate, the genome is deleted, fragmented, degraded, genetically inactivated, or any other physical, biological, chemical or structural manifestation, that prevents the virus from being transmissible or infecting any other cell or subject resulting in the clearance of the virus in vivo. In some cases, fragments of the viral genome may be detectable, however, the virus is incapable of replication, or infection etc.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The term “percent sequence identity” or having “a sequence identity” refers to the degree of identity between any given query sequence and a subject sequence.

As used herein, a “pharmaceutically acceptable” component/carrier etc. is one that is suitable for use with humans and/or animals without undue adverse side effects (such as toxicity, irritation, and allergic response) commensurate with a reasonable benefit/risk ratio.

The term “regulatory element” refers to any genetic element (e.g., polynucleotide sequence) that can exert a regulatory effect on the replication or expression (transcription or translation) of another genetic element. Common expression control sequences include promoters, polyadenylation (polyA) signals, transcription start site (TSS) sequences, transcription termination sequences, sequences encoding transcription factors (TFs), insulators (also called boundary elements), upstream regulatory domains, origins of replication, internal ribosome entry sites (IRES), enhancers, and the like.

The term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

To “treat” a disease as the term is used herein, means to reduce the frequency or severity of at least one sign or symptom of a disease or disorder experienced by a subject. Treatment of a disease or disorders includes the eradication of a virus.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) eradicating the virus; (2) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (3) inhibiting the state, disorder or condition, i.e., arresting, reducing or delaying the development of the disease or a relapse thereof (in case of maintenance treatment) or at least one clinical or subclinical symptom thereof; or (4) relieving the disease, i.e., causing regression of the state, disorder or condition or at least one of its clinical or subclinical symptoms. The benefit to an individual to be treated is either statistically significant or at least perceptible to the patient or to the physician.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

Where any amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

FIGS. 1A-1D show the excision of the exon1-intron1-exon2 region of the MOGS gene. Mannosyl-oligosaccharide glucosidase (MOGS, GC-1) is a transmembrane endoplasmic reticulum (ER) enzyme responsible for the initial step of remodeling of the N-linked glycans, i.e., removal of the first glucose residue, what allows further trimming and maturation of glycoproteins (FIG. 1A). The human MOGS gene is located on chromosome 2 (2p13.1); it is 4326 bp long and has a total of 4 exons (FIGS. 1B, 1C). We have designed a pair of guide RNAs targeting promoter and intron2 regions of the human MOGS gene. Successful cleavage at the target sites will lead to the deletion of the 1187bp long segment of DNA spanning exon1-intron 1-exon2 and containing the MOGS start codons (FIG. 1D) resulting in the knockout of MOGS expression. Figure disclosed SEQ ID NO: 1.

FIGS. 2A-2E demonstrate the CRISPR mediated MOGS gene knockout in TZM-bl cells. TZM-bl cells were transfected with in pX601-no-gRNA (control) or pX601-MOGS-A/MOGS-B plasmid together with pKLV-BFP (to provide selection marker) at ratio 10:1, then were selected for two weeks with 1 μg/ml puromycin and clonally expanded. Genomic DNA was extracted from one control and two MOGS knockout single-cell clones and subjected to PCRs specific to the exon1-intron1-exon2 region of the MOGS gene. Gel agarose electrophoresis confirmed the presence of CRISPR-Cas9 induced, double-cleaved/end-joined truncated amplicons in pX601-MOGS-A/MOGS-B treated clones (FIG. 2A). Truncated PCR products were verified by Sanger sequencing shown in (FIG. 2B). Figure discloses SEQ ID NOS 2 and 2, respectively, in order of appearance. Representative alignment of the sequencing results from TZM-bl single-cell clones (FIG. 2C; SEQ ID NOS 3, 4, and 4, respectively, in order of appearance.) gRNAs target sequences are highlighted in green, PAMs in red. Lack of MOGS expression was in knockout single-cell clones was confirmed or mRNA and protein level by RT-PCR (FIG. 2D) and Western blot (FIG. 2E), respectively.

FIGS. 3A-3D show MOGS knockout results in hyperglycosylated envelope and defective progeny virus. One WT and two MOGS knockout (clone2 and clone 3) TZM-bl single-cell clones were infected with CCR5-tropic HIV-1NL4-3-BAL-GFP reporter virus at MOI 0.25 (FIG. 3A). Supernatants from infected cells were collected every day for a total of 3 days and tested for infectivity by incubation with highly susceptible to HIV infection, HuTR5 cell line. After 48 h incubation, HutR5 cells were fixed with paraformaldehyde and analyzed for GFP expression by flow cytometry (FIG. 3B). The levels of the virus in supernatants used for secondary infection experiment were quantified by HIV Gag p24 ELISA (FIG. 3C) Additionally, at 48 h time point, some of the TZM-bl cells were harvested, and protein lysates were examined for HIV gp160 and p55 proteins expression by Western Blot (FIG. 3D).

FIGS. 4A-4C show MOGS gene knockout in T-lymphoid cells (Jurkat). FIG. 4A: Sanger sequencing results are shown, verifying the presence of InDel mutations at the gRNA target sites at both genomic loci (complete biallelic knockout). Figure discloses SEQ ID NOS 5-9, respectively, in order of appearance. Sequences alignment in FIG. 4B. Figure discloses SEQ ID NOS 10-14, respectively, in order of appearance. FIG. 4C: Western blot conducted, verified the lack of MOGS expression in knockout clones.

FIGS. 5A-5D show progeny virus released from MOGS″ T-cells. FIG. 5A: One WT and two MOGS knockout (clone5 and clone 13) Jurkat single-cell clones were infected with CCR5-tropic HIV-1NL4-3-BAL-GFP reporter virus at MOI 0.25 (FIG. 5A). After 48 h, supernatants from infected cells were collected and tested for infectivity by incubation with highly susceptible to HIV infection, HuTR5 cell line. After 48 h incubation, HutR5 cells were fixed with paraformaldehyde and analyzed for GFP expression by flow cytometry (FIG. 5B). The levels of the virus in supernatants used for secondary infection experiment were quantified by HIV Gag p24 ELISA (FIG. 5C). Additionally, Jurkat cells were harvested, and protein lysates were examined for HIV gp160, p55, and MOGS proteins expression by Western Blot (FIG. 5D). Glycosidase inhibitor, castanospermine, used at high 10 ug/ml concentration, showed very modest effects on progeny virus infectivity (FIG. 5B) and processing of gp160 (FIG. 5D).

FIG. 6 is a schematic representation showing CRISPR-Cas9 mediated knockout of MOGS gene expression in the host cells has potent antiviral activity. Lack of proper remodeling of glycan chains, decorating the viral envelope, results in non-infective progeny virions and breaks the infection cycle of the HIV-1.

FIGS. 7A-7F show the CRISPR-mediated MOGS gene knockout in a cell model. FIG. 7E discloses “ATGCCTCAGCCCTAAACAACTGCCTGTATAACC” as SEQ ID NO: 2. FIG. 7F discloses SEQ ID NOS 3-4, and 4, respectively, in order of appearance.

FIGS. 8A-8D demonstrate that editing MOGS affects infectivity of HIV.

FIGS. 9A-9F demonstrate that sequential treatment of T cell lines with CRISPR targeting MOGS and HIV-1 extinguishes secondary viral infection. FIG. 9A: Jurkat cells were electroporated with recombinant SaCas9 and synthetic gRNA targeting MOGS gene or control and then infected with HIV-Bal-GFP. The percentages of GFP positive (-infected) cells were detected by flow cytometry 2 days later. FIG. 9B: The HIV levels in the supernatants were quantified by HIV-1 gag p24 Elisa assay. FIG. 9C: The protein cell lysates were analyzed by Western Blot with antibodies as indicated. FIG. 9D: The Jurkat cells'supernatants from B. were used to infect HuTR5 cells (secondary infections). The next day infected cells were electroporated with recombinant SaCas9 and synthetic gRNAs targeting LTR/GagD or control. After another 2 days the number of infected, GFP positive cells was checked by flow cytometry. FIG. 9E: The HIV levels in the supernatants were quantified by HIV-1 gag p24 Elisa assay. FIG. 9F: The protein cell lysates were analyzed by Western Blot with antibodies as indicated.

FIGS. 10A-10F demonstrate CRISPR-Cas9 mediated MOGS gene knockout in TZM-bl cell clones. FIG. 10A: The human MOGS gene is located on chromosome 2 (2p13.1), it is 4326 bp long with 4 exons. A pair of guide RNAs are designed targeting promoter and intron2 regions of the MOGS gene. Successful cleavage at the target sites will lead to the deletion of the 1187bp long segment of DNA spanning exon1-intron1-exon2 which containing the MOGS start codons and result in the knockout of MOGS expression. FIG. 10B: The gene-specific PCR and FIG. 10C: the Sanger sequence confirmed CRISPR-Cas9 induced double-cleaved/end-joined truncations in the two pX601-MOGSAB treated TZM-bl cell clones. FIG. 10D: The alignment of the sequencing results from TZM-bi single-cell clones with wild-type MOGS gene were shown, and the gRNAs target sequences are highlighted in green, PAMs in red. FIG. 10E: The mRNA expressions and FIG. 10F: the protein expressions of MOGS gene were depleted in the two knockout TZM-bl cell clones verified by RT-PCR and Western blot respectively. FIG. 10C discloses “ATGCCTCAGCCCTAAACAACTGCCTGTATAACC” as SEQ ID NO: 2. FIG. 10D discloses SEQ ID NOS 3-4, and 4, respectively, in order of appearance.

FIGS. 11A-11D demonstrate the effect of CRISPR-Cas9 mediated MOGS gene knockout on the HIV replication in TZM-bl cells. FIG. 11A: To determine the infection efficiency of HIV-Bal on the MOGS gene knockout TZM-bl cell clones, the percentage of GFP positive TZM-bl cells were detected by FACS at different time after the viral infection. FIG. 11B: The wild-type and the MOGS gene knockout TZM-bl cells were pelleted at day 2 after HIV-Bal infection. The cell lysates were analyzed by Western Blot with antibodies as indicated, while HIV-p160 showed a higher molecular weight in the MOGS knockout TZM-bl cell clones. FIG. 11C: The supernatants of HIV-Bal infected TZM-bl cells were collected at the different time. The HIV viral amounts in the supernatants were detected by p24 Elisa assay. FIG. 11D: The TZM-bl supernatants collected at different time were used to incubate with HuTR5 cells, the GFP positive percentages of HuTR5 cells were detected by FACS after 2 days incubation.

FIGS. 12A-12D demonstrate the effect of CRISPR-Cas9 mediated MOGS gene knockout on the HIV replication in Jurkat cells. FIG. 12A: To determine the effect of MOGS gene knockout and MOGS inhibitor Castanospermine on the infection efficiency of HIV-Bal on the Jurkat cells, Jurkat cells were treated with or without 10 μg/mL Castanospermine and infected with HIV-Bal, the percentage of GFP positive cells were detected by FACS 2 days later. FIG. 12B: The Jurkat cells in (FIG. 12A) were pelleted at 2 days after HIV-Bal infection. The cell lysates were analyzed by Western Blot with antibodies as indicated, while HIV-p160 showed a higher molecular weight in the MOGS knockout Jurkat cell clones. FIG. 12C: The supernatants of HIV-Bal infected Jurkat cells in (FIG. 12A) were collected at Day2 after infection. The HIV viral amounts in the supernatants were detected by p24 Elisa assay. FIG. 12D: The Jurkat supernatants were used to incubate with HuTR5 cells, the GFP positive percentages of HuTR5 cells were detected by FACS 2 days later.

FIGS. 13A-13D demonstrate the effect of CRISPR-Cas9 mediated MOGS gene knockdown on the HIV replication in primary CD4⁺ T cells. FIG. 13A: Primary CD4⁺ T cells isolated from donated blood were electroporated by RNP with Cas9 and 4 different gRNAs. To verify the CRISPR-Cas9 mediated knock-down of MOGS, the cell lysates of primary CD4 T cells were analyzed by Western Blot with antibodies as indicated. FIG. 13B: To determine the infection efficiency of HIV-Bal on the MOGS gene knockdown primary CD4⁺ T cells, the HIV DNA copies were detected by digital PCR at day 2 after the viral infection. FIG. 13C: The supernatants of HIV-Bal infected primary CD4⁺ T cells were collected at Day 2 after infection. The HIV viral amounts in the supernatants were detected by p24 Elisa assay. FIG. 13D: The primary CD4⁺ T cells supernatants were used to incubate with HuTR5 cells, the HIV DNA copies in HuTR5 cells were detected by digital PCR after 2 days incubation.

FIGS. 14A-14F demonstrate the effect of CRISPR-Cas9 targeting MOGS gene and HIV LTR/GagD on the HIV replication in cultured T cells. FIG. 14A: Jurkat cells were electroporated with SaCas9 and gRNA targeting MOGS gene or control and infected with HIV-Bal-GFP, the percentage of GFP positive cells were detected by Guava 2 days later. FIG. 14B: The supernatants of HIV-Bal infected Jurkat cells in (FIG. 14A) were collected at Day2 after infection. The HIV viral amounts in the supernatants were detected by p24 Elisa assay. FIG. 14C: The Jurkat cells in (FIG. 14A) were pelleted at 2days after HIV-Bal infection. The cell lysates were analyzed by Western Blot with antibodies as indicated. FIG. 14D: The Jurkat supernatants in (FIG. 14A) were used to incubate with HuTR5 cells, and electroporated with SaCas9 and gRNAs targeting LTR/GagD or control at next day, the GFP positive percentages of HuTR5 cells were detected by Guava 2 days after incubation. FIG. 14E: The supernatants of HuTR5 cells in (FIG. 14D) were collected at Day2 after incubation. The HIV viral amounts in the supernatants were detected by p24 Elisa assay. FIG. 14F: The HuTR5 cells in (FIG. 15D) were pelleted at 2days after incubation. The cell lysates were analyzed by Western Blot with antibodies as indicated.

FIGS. 15A-15D demonstrate the combined effects of CRISPR-MOGS and CRISPR-HIV treatment on the infectivity of progeny virions derived from ex vivo treated patient-derived CD4 T cells. FIG. 15A: Primary HIV patient-derived CD4 T cells were electroporated with RNP-CRISPR complexes targeting the MOGS gene, HIV genome, or with a mixture of both. In addition, a subset of cells was electroporated with Cas9 only (no gRNAs) for the control. After three days, the cells were collected for genomic DNA, and protein analysis and supernatants were used in secondary infections. ddPCR quantification of viral DNA levels in the cells. FIG. 15B: HIV-1 Gag p24 ELISA results for the supernatants collected from CRISPR treated cells. FIG. 15C: HIV-1 Gag p24 ELISA results for the supernatants collected from secondary infections in HutR5 T-lymphoid cell line. The values were normalized to virus levels detected in supernatants from CRISPR treated primary patient-derived cells. FIG. 15D: Western blot analysis of the protein lysates prepared from CRISPR treated primary patient-derived cells.

FIGS. 16A-16D demonstrate the CRISPR-Cas9 mediated MOGS gene excision in TZM-bl cells. FIG. 16A: A pair of guide RNAs are designed targeting promoter and intron2 regions of the MOGS gene. Successful cleavage at the target sites will lead to the deletion of the 1187bp long segment of DNA spanning exon1-intron1-exon2 which containing the MOGS start codons. Figure discloses SEQ ID NO: 1. FIG. 16B: The plasmid of pX601-MOGSAB contains expression cassettes of Cas9 and two guide RNAs targeting MOGS gene. FIG. 16C: The plasmid of pX601-MOGSAB was transfected into TZM-bl cells. After 3days and 6days, the expressing of SaCas9 and guide RNAs of MOGSA and B were confirmed by reverse-transcription PCR. FIG. 16D: The excision of MOGS gene was detected by PCR.

FIGS. 17A-17C demonstrate the PCR and Sanger sequencing for predicted off target region of gRNAs MOGSA/B. FIG. 17A: The five predicted off target region was PCR amplified in the genomic DNA obtained from wildtype TZM-bl cells and two MOGS knockout clones. FIG. 17B: The PCR products of A was purified and analyzed by sanger sequencing. Figure discloses SEQ ID NOS 15-16, 15-17, 16, 18-19, 18-19, 18-21, 20-21, 20-23, 22-23, 22-25, 24-25, and 24-25, respectively, in order of appearance.

FIGS. 18A-18D demonstrate the CRISPR-Cas9 mediated MOGS gene knockout in Jurkat cells. FIG. 18A: One guide RNA is designed to target the exon 2 region of MOGS gene. Successful cleavage at the target sites will make the insertion or deletion and result in the knockout of MOGS expression. Figure discloses SEQ ID NO: 1. FIG. 18B: Jurkat cells were electroporated with synthetic gRNA and recombinant Cas9 protein and then clonally expanded and detected for the MOGS expression by Western blot. FIG. 18C: One Jurkat WT control and two MOGS knockout single-cell clones were selected for further studies. The CRISPR-Cas9 induced specific indel mutations at the gRNA target sites in both genomic loci were confirmed by gene-specific PCR and Sanger sequence. The alignment of the sequencing results were shown with the gRNAs target sequences highlighted in green and PAMs in red. Figure discloses SEQ ID NOS 5-11, 26, and 13-14, respectively, in order of appearance. FIG. 18D: The protein expressions of MOGS gene were depleted in the two knockout Jurkat cell clones verified by Western blot.

FIGS. 19A-19C demonstrate the CRISPR-Cas9 mediated MOGS gene editing in primary CD4⁺ T cells. FIG. 19A: Four guide RNAs were designed to target the exon 2 region of MOGS gene. Successful cleavage at the target sites will make the insertion or deletion and result in the knockout of MOGS expression. Primary CD4⁺ T cells were electroporated with synthetic gRNA and recombinant Cas9 protein and then detected for the MOGS by gene-specific PCR. FIG. 19B: The CRISPR-Cas9 induced knockout score of specific indel mutations at the four gRNA target sites were analyzed by Sanger sequence. FIG. 19C: To determine the infection efficiency of HIV-Bal on the MOGS gene edited primary CD4″ T cells, the percentage of GFP positive cells were detected by FACS at day 2 after the viral infection.

### DETAILED DESCRIPTION

Cells derived from patients with a rare genetic disease, type II congenital disorders of glycosylation (CDG-IIb), caused by mutations in the MOGS gene have a reduced ability to support a productive infection by various enveloped viruses including HIV-1.

In certain embodiments, a method of preventing or treating an HIV infection in a subject, comprising: administering to the subject a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target sequence in the integrated retroviral DNA and/or complementary to a target sequence of the MOGS gene.

Combinations of gRNAs are especially effective when expressed in multiplex fashion, that is, simultaneously in the same cell.

In certain embodiments, the at least one gRNA includes at least a first gRNA that is complementary to a target sequence in the integrated retroviral DNA; and a second gRNA that is complementary to another target sequence in the integrated retroviral DNA, whereby the intervening sequences between the two gRNAs are excised.

In certain embodiments, the gene editing complex comprises a first gRNA that is complementary to a target sequence in the integrated retroviral DNA; and a second gRNA that is complementary to another target sequence in the integrated retroviral DNA, whereby the intervening sequences between the two gRNAs are excised.

In certain embodiments, the at least one gRNA includes at least a first gRNA that is complementary to a target sequence in the MOGS gene; and a second gRNA that is complementary to another target sequence in the MOGS gene, whereby the intervening sequences between the two gRNAs are excised.

In certain embodiments, the gene editing complex comprises a first gRNA that is complementary to a target sequence in the MOGS gene; and a second gRNA that is complementary to another target sequence in the of the MOGS gene, whereby the intervening sequences between the two gRNAs are excised.

In embodiments, the compositions of the invention include nucleic acids encoding gene editing agents and at least one guide RNA (gRNA) that is complementary to a target sequence in a retrovirus, e.g. HIV. In embodiments, the gene editing agents comprise: Cre recombinases, CRISPR/Cas molecules, TALE transcriptional activators, Cas9 nucleases, nickases, transcriptional regulators, homologues, orthologs or combinations thereof.

Provided herein, in some embodiments, are methods and compositions comprising a CRISPR-associated (Cas) peptide or a nucleic acid sequence encoding the CRISPR-associated (Cas) peptide and a plurality of guide nucleic acids or a nucleic acid sequence encoding the plurality of guide nucleic acids. In some embodiments, compositions and methods described herein comprise 1, 2, 3, 4, 5, 6, or more than 6 gRNAs. In some embodiments, compositions and methods described herein comprise 1, 2, 3, 4, 5, 6, or more than 6 different gRNAs. In some embodiments, compositions and methods described herein comprise 4 or at least 4 different gRNAs.

In certain embodiments, the compositions of the disclosure include nucleic acids encoding gene editing agents and at least one guide RNA (gRNA) that is complementary to a target sequence in the MOGS gene. In certain embodiments, the gene editing agents comprise: Cre recombinases, CRISPR/Cas molecules, TALE transcriptional activators, Cas9 nucleases, nickases, transcriptional regulators, homologues, orthologs or combinations thereof. In certain embodiments, the retrovirus target sequences comprise coding sequences, noncoding sequences or combinations thereof. In certain embodiments, the guide nucleic acid sequences target one or more target sequenceS in the MOGS gene.

In certain embodiments, the compositions of the disclosure include nucleic acids encoding gene editing agents and at least one guide RNA (gRNA) that is complementary to a target sequence in a retrovirus, e.g. HIV type 1 or 2. In certain embodiments, the gene editing agents comprise: Cre recombinases, CRISPR/Cas molecules, TALE transcriptional activators, Cas9 nucleases, nickases, transcriptional regulators, homologues, orthologs or combinations thereof.

In some embodiments, different gRNAs target different sequences within the 5′-and/or 3′ long terminal repeat (LTR) genes. In some embodiments, the different gRNAs are complementary to different target sequences within the LTR gene. In some embodiments, a target sequence is within or near the LTR gene. In some embodiments, a region near the LTR gene comprises 1, 2, 3, 4. 5, 10, 15, 20, 25, 30, or 35 base positions surrounding the LTR gene.

In some embodiments, the different gRNAs target different sequences within the Gag gene. In some embodiments, the different gRNAs are complementary to different target sequences within the Gag gene. In some embodiments, a target sequence is within or near the Gag gene. In some embodiments, a region near the Gag gene comprises 1, 2, 3, 4. 5, 10, 15, 20, 25, 30, or 35 base positions surrounding the Gag gene.

In some embodiments, the different gRNAs target different sequences within the Pol gene. In some embodiments, the different gRNAs are complementary to different target sequences within the Pol gene. In some embodiments, a target sequence is within or near the Pol gene. In some embodiments, a region near the Pol gene comprises 1, 2, 3, 4. 5, 10, 15, 20, 25, 30, or 35 base positions surrounding the Pol gene.

In some embodiments, the different gRNAs target different sequences within the Pro gene. In some embodiments, the different gRNAs are complementary to different target sequences within the Pro gene. In some embodiments, a target sequence is within or near the Pro gene. In some embodiments, a region near the Pro gene comprises 1, 2, 3, 4. 5, 10, 15, 20, 25, 30, or 35 base positions surrounding the Pro gene.

In some embodiments, the different gRNAs target different sequences within the Env gene. In some embodiments, the different gRNAs are complementary to different target sequences within the Env gene. In some embodiments, a target sequence is within or near the Env gene. In some embodiments, a region near the Env gene comprises 1, 2, 3, 4. 5, 10, 15, 20, 25, 30, or 35 base positions surrounding the Env gene.

In certain embodiments, a composition comprises a viral vector encoding a gene editing agent and at least one guide RNA (gRNA) wherein the gRNA is complementary to a target nucleic acid sequence of an HIV gene sequence, comprising a coding and/or non-coding HIV gene sequences. In certain embodiments, the guide nucleic acid sequences target one or more HIV sequences comprising: structural gene sequences, enzymatic gene sequences, regulatory genes, accessory genes, transactivator gene sequences or combinations thereof. In certain embodiments, the guide nucleic acid sequences target one or more HIV sequences comprising: structural gene sequences, enzymatic gene sequences, regulatory genes, accessory genes, transactivator gene sequences or combinations thereof.

In certain embodiments, a composition comprises a viral vector encoding a gene editing agent and at least one guide RNA (gRNA) wherein the gRNA is complementary to a target nucleic acid sequence in the MOGS gene.

An example of MOGS sequences can be found at accession numbers:

ENSEMBL:ENSG00000115275; NCBI_Gene:7841;
RGD:732423; UniProtKB:Q13724 Gene ID: 7841.
Homo sapiens mannosyl-oligosaccharide
glucosidase (MOGS), transcript variant 1,
mRNA. Accession: NM 006302:
(SEQ ID NO: 147)

| | |
|---|---|
| 1 | gcaggcgctg gctggcaggt gtcgctaacc ggacggtggt |
| | cgccagggcg agaggcggga |
| 61 | gccggagagg tgaggcagga cccgggctcc actgccgcct |
| | ctccgagctc ttgtgacgcg |
| 121 | gacctcagtg ccaggatggc tcggggcgag cggcggcgcc |
| | gcgcagtgcc ggcagaggga |
| 181 | gtgcggacag ccgagagggc ggctcgggga ggccccgggc |
| | gacgggacgg ccggggcggc |
| 241 | gggccgcgta gcacggctgg aggagtggct ctggccgtcg |
| | tggtcctgtc tttggccctg |
| 301 | ggtatgtcgg ggcgctgggt gctggcgtgg taccgtgcgc |
| | ggcgggcggt cacgctgcac |
| 361 | tccgcgcctc ctgtgttgcc tgccgactcc tccagccccg |
| | ccgtggcccc ggacctcttc |
| 421 | tggggaacct accgccctca cgtctacttc ggcatgaaga |
| | cccgcagccc gaagcccctc |
| 481 | ctcaccggac tgatgtgggc gcagcagggc accaccccgg |
| | ggactcctaa gctcaggcac |
| 541 | acgtgtgagc agggggacgg tgtgggtccc tatggctggg |
| | agttccacga cggcctctcc |
| 601 | ttcgggcgcc aacacatcca ggatggggcc ttaaggctca |
| | ccactgagtt cgtcaagagg |
| 661 | cctgggggtc agcacggagg ggactggagc tggagagtga |
| | ctgtagagcc tcaggactca |
| 721 | ggtacttctg ccctcccttt ggtctccctg ttcttctatg |
| | tggtgacaga tggcaaggaa |
| 781 | gtcctactac cagaggttgg ggccaagggg cagttgaagt |
| | ttatcagtgg gcacaccagt |
| 841 | gaacttggtg acttccgctt tacacttttg ccaccaacca |
| | gtccagggga tacagccccc |
| 901 | aagtatggca gctacaatgt cttctggacc tccaacccag |
| | gactgcccct gctgacagag |
| 961 | atggtaaaga gtcgcctaaa tagctggttt cagcatcggc |
| | ccccaggggc cccccctgaa |
| 1021 | cgctacctcg gcttgccagg atccctgaag tgggaggaca |
| | gaggtccaag tgggcaaggg |
| 1081 | caggggcagt tcttgataca gcaggtgacc ctgaaaattc |
| | ccatttccat agagtttgtg |
| 1141 | tttgaatcag gcagtgccca ggcaggagga aatcaagccc |
| | tgccaagact ggcaggcagt |
| 1201 | ctactgaccc aggccctgga gagccatgct gaaggcttta |
| | gagagcgctt tgagaagacc |
| 1261 | ttccagctga aggagaaggg cctgagctct ggcgagcagg |
| | ttttgggtca ggctgccctc |
| 1321 | agcggcctcc ttggtggaat tggctacttc tacggacaag |
| | ggctggtatt gccagacatc |
| 1381 | ggggtggaag ggtctgagca gaaggtggac ccagccctct |
| | ttccacccgt acctcttttt |
| 1441 | acagcagtgc cctcccggtc attcttccca cgaggcttcc |
| | tttgggatga aggctttcac |
| 1501 | cagctggtgg ttcagcggtg ggatccctcc ctcacccggg |
| | aagcccttgg ccactggctg |
| 1561 | gggctgctaa atgctgatgg ctggattggg agggagcaga |
| | tactggggga tgaggcccga |
| 1621 | gcccgggtgc ctccagaatt cctagtacaa cgagcagtcc |
| | acgccaaccc cccaacccta |
| 1681 | cttttgcctg tagcccatat gctagaggtt ggtgaccctg |
| | acgacttggc tttcctccga |
| 1741 | aaggccttgc cccgcctgca tgcctggttt tcctggctcc |
| | atcagagcca ggcaggccca |
| 1801 | ctgccactat cttaccgctg gcggggacgg gaccctgcct |
| | taccaacctt actgaacccc |
| 1861 | aagaccctac cctctgggct ggatgactac ccccgggctt |
| | cacacccttc agtaaccgag |
| 1921 | cggcacctgg acctgcgatg ttgggtggca ctgggtgccc |
| | gtgtgctgac gcggctggca |
| 1981 | gagcatctgg gtgaggctga ggtagctgct gagctgggcc |
| | cactggctgc ctcactggag |
| 2041 | gcagcagaga gcctggatga gctgcactgg gccccagagc |
| | taggagtctt tgcagacttt |
| 2101 | gggaaccaca caaaagcagt acagctgaag cccaggcccc |
| | ctcaggggct cgttcgggtg |
| 2161 | gtgggtcggc cccaacctca actgcagtat gtagatgctc |
| | ttggctatgt cagtcttttt |
| 2221 | cccttgctgc tgcgactgct ggaccccacc tcatcccgcc |
| | ttgggcccct gctggacatt |
| 2281 | ctagccgaca gccgccatct ctggagcccc tttggtttac |
| | gctcccttgc agcctccagc |
| 2341 | tccttttatg gccagcgcaa ttcagagcat gatcccccct |
| | actggcgggg tgctgtgtgg |
| 2401 | ctcaatgtca actacctggc tttgggagca ctccaccact |
| | atgggcatct ggagggtcct |
| 2461 | caccaggctc gggctgccaa actccacggt gagctccgtg |
| | ccaacgtggt aggcaatgta |
| 2521 | tggcgccagt accaggctac aggctttctt tgggagcagt |
| | acagtgaccg cgatgggcga |
| 2581 | ggcatgggct gccgcccttt ccacggctgg accagccttg |
| | tcttactggc catggctgaa |
| 2641 | gactactgaa gggagggaga ggaggggagc caagacactc |
| | atgccactct ggctctgaag |
| 2701 | ggacaaaggc ttctggcttt tgcccccagc cccttggata |
| | ccagtaattC aaaccttcct |
| 2761 | catttcatct caggtgtctc cttgctgtca tcccacatag |
| | ccctggggtg aatgtgaatc |
| 2821 | cagagtctat ttttctaaat aaattggaaa aaacattttg |
| | aactcta |

(SEQ ID NO: 148)
MARCGERRRRAVPAEGVRTAERAARGCGPGRRDGRGGGPRSTAGGVALAV
VVLSLALGMSGRWVLAWYRARRAVTLHSAPPVLPADSSSPAVAPDLFWGT
YRPHVYFGMKTRSPKPLLTGLMWAQQGTTPGTPKLRHTCEQGDGVGPYGW
EFHDGLSFGRQHIQDGALRLTTEFVKRPGGQHGGDWSWRVTVEPQDSGTS
ALPLVSLFFYVVTDGKEVLLPEVGAKGQLKFISGHTSELGDFRFTLLPPT
SPGDTAPKYGSYNVFWTSNPGLPLLTEMVKSRLNSWFQHRPPGAPPERYL
GLPGSLKWEDRGPSGQGQGQFLIQQVTLKIPISIEFVFESGSAQAGGNQA
LPRLAGSLLTQALESHAEGFRERFEKTFQLKEKGLSSGEQVLGQAALSGL
LGGIGYFYGQGLVLPDIGVEGSEQKVDPALFPPVPLFTAVPSRSFFPRGF
LWDEGFHQLVVQRWDPSLTREALGHWLGLLNADGWIGREQILGDEARARV
PPEFLVQRAVHANPPTLLLPVAHMLEVGDPDDLAFLRKALPRLHAWFSWL
HQSQAGPLPLSYRWRGRDPALPTLLNPKTLPSGLDDYPRASHPSVTERHL
DLRCWVALGARVLTRLAEHLGEAEVAAELGPLAASLEAAESLDELHWAPE
LGVFADFGNHTKAVQLKPRPPQGLVRVVGRPQPQLQYVDALGYVSLFPLL
LRLLDPTSSRLGPLLDILADSRHLWSPFGLRSLAASSSFYGQRNSEHDPP
YWRGAVWLNVNYLALGALHHYGHLEGPHQARAAKLHGELRANVVGNVWRQ
YQATGFLWEQYSDRDGRGMGCRPFHGWTSLVLLAMAEDY

In certain embodiments, a viral vector comprises an adenovirus vector, an adeno-associated viral vector (AAV), or derivatives thereof. In some embodiments, the nucleic acids are configured to be packaged into an adeno-associated virus (AAV) vector. In some embodiments, the adeno-associated virus (AAV) vector is AAV2, AAV5, AAV6, AAV7, AAV8, or AAV9. In some embodiments, the adeno-associated virus (AAV) vector is AAV1, AAV2, AAV3, AAV4, AAV5, AAV6, AAV7, AAV8, AAV9, AAV10, AAV11, AAVDJ, or AAVDJ/8.

### Gene Editing Agents

Compositions of the disclosure include at least one gene editing agent, comprising CRISPR-associated nucleases such as Cas9 and Cas12a gRNAs, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- or exo-nucleases, or combinations thereof.

CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligonucleotide to hybridize to target and recruit the Cas/gRNA complex. See Chang et al., 2013, Cell Res. 23:465-472; Hwang et al., 2013, Nat. Biotechnol. 31:227-229; Xiao et al., 2013, Nucl. Acids Res. 1-11.

In general, the CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNase domains, protein-protein interaction domains, dimerization domains, as well as other domains. The mechanism through which CRISPR/Cas9-induced mutations inactivate the provirus can vary. For example, the mutation can affect proviral replication, and viral gene expression. The mutation can comprise one or more deletions. The size of the deletion can vary from a single nucleotide base pair to about 10,000 base pairs. In some embodiments, the deletion can include all or substantially all of the proviral sequence and/or the MOGS gene sequence.

CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligonucleotide to hybridize to target and recruit the Cas/gRNA complex. See Chang et al., 2013, Cell Res. 23:465-472; Hwang et al., 2013, Nat. Biotechnol. 31:227-229; Xiao et al., 2013, Nucl. Acids Res. 1-11.

The RNA-guided Cas9 biotechnology induces genome editing without detectable off-target effects. This technique takes advantage of the genome defense mechanisms in bacteria that CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). Cas9 belongs to the type II CRISPR/Cas system and has strong endonuclease activity to cut target DNA.

In certain embodiments, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas9 protein or fragment thereof. In other embodiments, the CRISPR/Cas-like protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In one embodiment, the RNA-guided endonuclease is derived from a type II CRISPR/Cas system. The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA: tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately. Therefore, the Cas9 gRNA technology requires the expression of the Cas9 protein and gRNA, which then form a gene editing complex at the specific target DNA binding site within the target genome and inflict cleavage/mutation of the target DNA.

However, the present disclosure is not limited to the use of Cas9-mediated gene editing. Rather, the present disclosure encompasses the use of other CRISPR-associated peptides, which can be targeted to a targeted sequence using a gRNA and can edit to target site of interest. For example, in some embodiments, the disclosure utilizes Cas12a (also known as Cpf1) to edit the target site of interest.

Engineered CRISPR systems generally contain two components: a guide RNA (gRNA or sgRNA) and a CRISPR-associated endonuclease (Cas protein). In nature, CRISPR/CRISPR-associated (Cas) systems provide bacteria and archaea with adaptive immunity against viruses and plasmids by using CRISPR RNAs (crRNAs) to guide the silencing of invading nucleic acids. The CRISPR-Cas is a RNA-mediated adaptive defense system that relies on small RNA molecules for sequence-specific detection and silencing of foreign nucleic acids. CRISPR/Cas systems are composed of cas genes organized in operon(s) and CRISPR array(s) consisting of genome-targeting sequences (called spacers).

As described herein, CRISPR-Cas systems generally refer to an enzyme system that includes a guide RNA sequence that contains a nucleotide sequence complementary or substantially complementary to a region of a target polynucleotide, and a protein with nuclease activity. CRISPR-Cas systems include Type I CRISPR-Cas system, Type II CRISPR-Cas system, Type III CRISPR-Cas system, and derivatives thereof. CRISPR-Cas systems include engineered and/or programmed nuclease systems derived from naturally accruing CRISPR-Cas systems. In certain embodiments, CRISPR-Cas systems contain engineered and/or mutated Cas proteins. In some embodiments, nucleases generally refer to enzymes capable of cleaving the phosphodiester bonds between the nucleotide subunits of nucleic acids. In some embodiments, endonucleases are generally capable of cleaving the phosphodiester bond within a polynucleotide chain. Nickases refer to endonucleases that cleave only a single strand of a DNA duplex.

In some embodiments, the CRISPR/Cas system used herein can be a type I, a type II, or a type III system. Non-limiting examples of suitable CRISPR/Cas proteins include Cas3, Cas4, Cas5, Cas5e (or CasD), Cas6, Cas6e, Casof, Cas7, Cas8al, Cas8a2, Cas8b, Cas8c, Cas9, Cas10, Cas10d, CasF, CasG, CasH, CasX, Caso, Csyl, Csy2, Csy3, Csel (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. By way of further example, in some embodiments, the CRISPR-Cas protein is a C as1, Cas1B, Cas2, Cas3, Cas4, Cas5, Cash, Cas7, Cas8, Cas10, Csyl, Csy2, Csy3, Csel, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx16, CsaX, Csx3, Csx1, Csx15, Csf1, Csf2, Csf3, Csf4, Cas9, Cas12 (e.g., Cas12a, Cas12b, Cas12c, Cas12d, Cas12k, Cas12j/Caso, Cas12L etc.), Cas13 (e.g., Cas13a, Cas13b (such as Cas13b-t1, Cas13b-t2, Cas13b-t3), Cas13c, Cas13d, etc.), Cas14, CasX, CasY, or an engineered form of the Cas protein. In some embodiments, the CRISPR/Cas protein or endonuclease is Cas9. In some embodiments, the CRISPR/Cas protein or endonuclease is Cas12. In certain embodiments, the Cas12 polypeptide is Cas12a, Cas12b, Cas12c, Cas12d, Cas12e, Cas12g, Cas12h, Cas12i, Cas12L or Cas12J. In some embodiments, the CRISPR/Cas protein or endonuclease is CasX. In some embodiments, the CRISPR/Cas protein or endonuclease is CasY. In some embodiments, the CRISPR/Cas protein or endonuclease is Cas@.

In some embodiments, the Cas9 protein can be from or derived from: Staphylococcus aureus, Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Fine goldia magna, Natranaerobius thermophilus, Pelotomaculum the rmopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In some embodiments, the composition comprises a CRISPR-associated (Cas) protein, or functional fragment or derivative thereof. In some embodiments, the Cas protein is an endonuclease, including but not limited to the Cas9 nuclease. In some embodiments, the Cas9 protein comprises an amino acid sequence identical to the wild type Streptococcus pyogenes or Staphylococcus aureus Cas9 amino acid sequence. In some embodiments, the Cas protein comprises the amino acid sequence of a Cas protein from other species, for example other Streptococcus species, such as thermophilus; Pseudomonas aeruginosa, Escherichia coli, or other sequenced bacteria genomes and archaea, or other prokaryotic microorganisms. Other Cas proteins, useful for the present disclosure, known or can be identified, using methods known in the art (see e.g., Esvelt et al., 2013, Nature Methods, 10:1116-1121). In some embodiments, the Cas protein comprises a modified amino acid sequence, as compared to its natural source. CRISPR/Cas proteins comprise at least one RNA recognition and/or RNA binding domain. RNA recognition and/or RNA binding domains interact with guide RNAs (gRNAs). CRISPR/Cas proteins can also comprise nuclease domains (i.e., DNase or RNase domains), DNA binding domains, helicase domains, RNAse domains, protein-protein interaction domains, dimerization domains, as well as other domains.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the Cas protein. The CRISPR/Cas-like protein can also be truncated or modified to optimize the activity of the effector domain of the Cas protein.

In some embodiments, the CRISPR/Cas-like protein can be derived from a wild type Cas protein or fragment thereof. In some embodiments, the CRISPR/Cas-like protein is a modified Cas9 protein. For example, the amino acid sequence of the Cas9 protein can be modified to alter one or more properties (e.g., nuclease activity, affinity, stability, etc.) of the protein relative to wild-type or another Cas protein. Alternatively, domains of the Cas9 protein not involved in RNA-guided cleavage can be eliminated from the protein such that the modified Cas9 protein is smaller than the wild-type Cas9 protein.

The disclosed CRISPR-Cas compositions should also be construed to include any form of a protein having substantial homology to a Cas protein (e.g., Cas9, saCas9, Cas9 protein) disclosed herein. In some embodiments, a protein which is “substantially homologous” is about 50% homologous, about 70% homologous, about 80% homologous, about 90% homologous, about 95% homologous, or about 99% homologous to amino acid sequence of a Cas protein disclosed herein. The Cas9 can be an orthologous. Six smaller Cas9 orthologues have been used and reports have shown that Cas9 from Staphylococcus aureus (SaCas9) can edit the genome with efficiencies similar to those of SpCas9, while being more than 1 kilobase shorter.

In some embodiments, the composition comprises a CRISPR-associated (Cas) peptide, or functional fragment or derivative thereof. In certain embodiments, the Cas peptide is an endonuclease, including but not limited to the Cas9 nuclease. In some embodiments, the Cas9 peptide comprises an amino acid sequence identical to the wild type Streptococcus pyogenesCas9 amino acid sequence. In some embodiments, the Cas peptide may comprise the amino acid sequence of a Cas protein from other species, for example other Streptococcus species, such as thermophilus; Psuedomonas aeruginosa, Escherichia coli, or other sequenced bacteria genomes and archaea, or other prokaryotic microogranisms. Other Cas peptides, useful for the present disclosure, known or can be identified, using methods known in the art (see e.g., Esvelt et al., 2013, Nature Methods, 10:1116-1121). In certain embodiments, the Cas peptide may comprise a modified amino acid sequence, as compared to its natural source. For example, in some embodiments, the wild type Streptococcus pyogenes Cas9 sequence can be modified. In certain embodiments, the amino acid sequence can be codon optimized for efficient expression in human cells (i.e., “humanized) or in a species of interest. A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as PX330 or PX260 from Addgene (Cambridge, MA). In some embodiments, the Cas9 endonuclease can have an amino acid sequence that is a variant or a fragment of any of the Cas9 endonuclease sequences of Genbank accession numbers KM099231.1 GL669193757; KM099232.1 GL669193761; or KM099233.1 GL669193765 or Cas9 amino acid sequence of PX330 or PX260 (Addgene, Cambridge, MA).

The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution).

In certain embodiments, the Cas peptide is a mutant Cas9, wherein the mutant Cas9 reduces the off-target effects, as compared to wild-type Cas9. In some embodiments, the mutant Cas9 is a Streptococcus pyogenes Cas9 (SpCas9) variant.

In some embodiments, SpCas9 variants comprise one or more point mutations, including, but not limited to R780A, K810A, K848A, K855A, H982A, K1003A, and R1060A (Slaymaker et al., 2016, Science, 351 (6268): 84-88). In some embodiments, SpCas9 variants comprise D1135E point mutation (Kleinstiver et al., 2015, Nature, 523 (7561): 481-485). In some embodiments, SpCas9 variants comprise one or more point mutations, including, but not limited to N497A, R661A, Q695A, Q926A, D1135E, L169A, and Y450A (Kleinstiver et al., 2016, Nature, doi: 10.1038/nature16526). In some embodiments, SpCas9 variants comprise one or more point mutations, including but not limited to M495A, M694A, and M698A. Y450 is involved with hydrophobic base pair stacking. N497, R661, Q695, Q926 are involved with residue to base hydrogen bonding contributing to off-target effects. N497 hydrogen bonding through peptide backbone. L169A is involved with hydrophobic base pair stacking. M495A, M694A, and H698A are involved with hydrophobic base pair stacking.

In some embodiments, SpCas9 variants comprise one or more point mutations at one or more of the following residues: R780, K810, K848, K855, H982, K1003, R1060, D1135, N497, R661, Q695, Q926, L169, Y450, M495, M694, and M698. In some embodiments, SpCas9 variants comprise one or more point mutations selected from the group of: R780A, K810A, K848A, K855A, H982A, K1003A, R1060A, D1135E, N497A, R661A, Q695A, Q926A, L169A, Y450A, M495A, M694A, and M698A.

In some embodiments, the SpCas9 variant comprises the point mutations, relative to wildtype SpCas9, of N497A, R661A, Q695A, and Q926A. In some embodiments, the SpCas9 variant comprises the point mutations, relative to wildtype SpCas9, of N497A, R661A, Q695A, Q926A, and D1135E. In some embodiments, the SpCas9 variant comprises the point mutations, relative to wildtype SpCas9, of N497A, R661A, Q695A, Q926A, and L169A. In some embodiments, the SpCas9 variant comprises the point mutations, relative to wildtype SpCas9, of N497A, R661A, Q695A, Q926A, and Y450A. In some embodiments, the SpCas9 variant comprises the point mutations, relative to wildtype SpCas9, of N497A, R661A, Q695A, Q926A, and M495A. In some embodiments, the SpCas9 variant comprises the point mutations, relative to wildtype SpCas9, of N497A, R661A, Q695A, Q926A, and M694A. In some embodiments, the SpCas9 variant comprises the point mutations, relative to wildtype SpCas9, of N497A, R661A, Q695A, Q926A, and H698A. In some embodiments, the SpCas9 variant comprises the point mutations, relative to wildtype SpCas9, of N497A, R661A, Q695A, Q926A, D1135E, and L169A. In some embodiments, the SpCas9 variant comprises the point mutations, relative to wildtype SpCas9, of N497A, R661A, Q695A, Q926A, D1135E, and Y450A. In some embodiments, the SpCas9 variant comprises the point mutations, relative to wildtype SpCas9, of N497A, R661A, Q695A, Q926A, D1135E, and M495A. In some embodiments, the SpCas9 variant comprises the point mutations, relative to wildtype SpCas9, of N497A, R661A, Q695A, Q926A, D1135E, and M694A. In some embodiments, the SpCas9 variant comprises the point mutations, relative to wildtype SpCas9, of N497A, R661A, Q695A, Q926A, D1135E, and M698A.

In some embodiments, the SpCas9 variant comprises the point mutations, relative to wildtype SpCas9, of R661A, Q695A, and Q926A. In some embodiments, the SpCas9 variant comprises the point mutations, relative to wildtype SpCas9, of R661A, Q695A, Q926A, and D1135E. In some embodiments, the SpCas9 variant comprises the point mutations, relative to wildtype SpCas9, of R661A, Q695A, Q926A, and L169A. In some embodiments, the SpCas9 variant comprises the point mutations, relative to wildtype SpCas9, of R661A, Q695A, Q926A, and Y450A. In some embodiments, the SpCas9 variant comprises the point mutations, relative to wildtype SpCas9, of R661A, Q695A, Q926A, and M495A. In some embodiments, the SpCas9 variant comprises the point mutations, relative to wildtype SpCas9, of R661A, Q695A, Q926A, and M694A. In some embodiments, the SpCas9 variant comprises the point mutations, relative to wildtype SpCas9, of R661A, Q695A, Q926A, and H698A. In some embodiments, the SpCas9 variant comprises the point mutations, relative to wildtype SpCas9, of R661A, Q695A, Q926A, D1135E, and L169A. In some embodiments, the SpCas9 variant comprises the point mutations, relative to wildtype SpCas9, of R661A, Q695A, Q926A, D1135E, and Y450A. In some embodiments, the SpCas9 variant comprises the point mutations, relative to wildtype SpCas9, of R661A, Q695A, Q926A, D1135E, and M495A. In some embodiments, the SpCas9 variant comprises the point mutations, relative to wildtype SpCas9, of R661A, Q695A, Q926A, D1135E, and M694A. In some embodiments, the SpCas9 variant comprises the point mutations, relative to wildtype SpCas9, of R661A, Q695A, Q926A, D1135E, and M698A.

In some embodiments, the mutant Cas9 comprises one or more mutations that alter PAM specificity (Kleinstiver et al., 2015, Nature, 523 (7561): 481-485; Kleinstiver et al., 2015, Nat Biotechnol, 33 (12): 1293-1298). In some embodiments, the mutant Cas9 comprises one or more mutations that alter the catalytic activity of Cas9, including but not limited to D10A in RuvC and H840A in HNH (Cong et al., 2013; Science 339:919-823, Gasiubas et al., 2012; PNAS 109: E2579-2586 Jinek et al., 2012; Science 337:816-821).

In addition to the wild type and variant Cas9 endonucleases described, embodiments of the disclosure also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI: 10.1126/science.aad5227).

In certain embodiments, three variants found to have the best cleavage efficiency and fewest off-target effects: SpCas9 (K855A), SpCas9 (K810A/K1003A/R1060A) (a.k.a. eSpCas9 1.0), and SpCas9 (K848A/K1003A/R1060A) (a.k.a. eSPCas9 1.1) are employed in the compositions. The disclosure is by no means limited to these variants, and also encompasses all Cas9 variants (Slaymaker, I.M. et al. (2015)). The present disclosure also includes another type of enhanced specificity Cas9 variant, “high fidelity” spCas9 variants (HF-Cas9). Examples of high fidelity variants include SpCas9-HF1 (N497A/R661A/Q695A/Q926A), SpCas9-HF2 (N497A/R661A/Q695A/Q926A/D1135E), SpCas9-HF3 (N497A/R661A/Q695A/Q926A/L169A), SpCas9-HF4 (N497A/R661A/Q695A/Q926A/Y450A). Also included are all SpCas9 variants bearing all possible single, double, triple and quadruple combinations of N497A, R661A, Q695A, Q926A or any other substitutions (Kleinstiver, B. P. et al., 2016, Nature. DOI: 10.1038/nature16526).

Accordingly, in certain embodiments, a Cas9 variant comprises a human-optimized Cas9; a nickase mutant Cas9; saCas9; enhanced-fidelity SaCas9 (efSaCas9); SpCas9 (K855a); SpCas9 (K810A/K1003A/r1060A); SpCas9 (K848A/K1003A/R1060A); SpCas9 N497A, R661A, Q695A, Q926A; SpCas9 N497A, R661A, Q695A, Q926A, D1135E; SpCas9 N497A, R661A, Q695A, Q926A L169A; SpCas9 N497A, R661A, Q695A, Q926A Y450A; SpCas9 N497A, R661A, Q695A, Q926A M495A; SpCas9 N497A, R661A, Q695A, Q926A M694A; SpCas9 N497A, R661A, Q695A, Q926A H698A; SpCas9 N497A, R661A, Q695A, Q926A, D1135E, L169A; SpCas9 N497A, R661A, Q695A, Q926A, D1135E, Y450A; SpCas9 N497A, R661A, Q695A, Q926A, D1135E, M495A; SpCas9 N497A, R661A, Q695A, Q926A, D1135E, M694A; SpCas9 N497A, R661A, Q695A, Q926A, D1135E, M698A; SpCas9 R661A, Q695A, Q926A; SpCas9 R661A, Q695A, Q926A, D1135E; SpCas9 R661A, Q695A, Q926A, L169A; SpCas9 R661A, Q695A, Q926A Y450A; SpCas9 R661A, Q695A, Q926A M495A; SpCas9 R661A, Q695A, Q926A M694A; SpCas9 R661A, Q695A, Q926A H698A; SpCas9 R661A, Q695A, Q926A D1135E L169A; SpCas9 R661A, Q695A, Q926A D1135E Y450A; SpCas9 R661A, Q695A, Q926A D1135E M495A; or SpCas9 R661A, Q695A, Q926A, D1135E or M694A.

As used herein, the term “Cas” is meant to include all Cas molecules comprising variants, mutants, orthologues, high-fidelity variants and the like.

However, the present disclosure is not limited to the use of Cas9-mediated gene editing. Rather, the present disclosure encompasses the use of other CRISPR-associated peptides, which can be targeted to a targeted sequence using a gRNA and can edit to target site of interest. For example, in some embodiments, the disclosure utilizes Cpfl to edit the target site of interest. Cpf1 is a single crRNA-guided, class 2 CRISPR effector protein which can effectively edit target DNA sequences in human cells. Exemplary Cpfl includes, but is not limited to, Acidaminococcus sp. Cpf1 (AsCpf1) and Lachospiraceae bacterium Cpf1 (LbCpf1).

The disclosure should also be construed to include any form of a peptide having substantial homology to a Cas peptide (e.g., Cas9) disclosed herein. Preferably, a peptide which is “substantially homologous” is about 50% homologous, more preferably about 70% homologous, even more preferably about 80% homologous, more preferably about 90% homologous, even more preferably, about 95% homologous, and even more preferably about 99% homologous to amino acid sequence of a Cas peptide disclosed herein.

The guide RNA sequence can be a sense or anti-sense sequence. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. For example, Cas9 from S. thermophilus requires 5′-NNAGAA for CRISPR 1 and 5′-NGGNG for CRISPR3) and Neiseria menigiditis requires 5′-NNNNGATT). The specific sequence of the guide RNA may vary, but, regardless of the sequence, useful guide RNA sequences will be those that minimize off-target effects while achieving high efficiency and complete ablation of the HIV. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. Useful selection methods identify regions having extremely low homology between the foreign viral genome and host cellular genome including endogenous retroviral DNA, include bioinformatic screening using 12-bp+NGG target-selection criteria to exclude off-target human transcriptome or (even rarely) untranslated-genomic sites.

### Guide RNAs

The guide RNA sequences can be configured as a single sequence or as a combination of one or more different sequences, e.g., a multiplex configuration. Multiplex configurations can include combinations of two, three, four, five, six, seven, eight, nine, ten, or more different guide RNAs.

When the compositions are administered in an expression vector, the guide RNAs can be encoded by a single vector. Alternatively, multiple vectors can be engineered to each include two or more different guide RNAs. Useful configurations will result in the excision of viral sequences between cleavage sites resulting in the ablation of HIV genome or HIV protein expression or the MOGS gene. Thus, the use of two or more different guide RNAs promotes excision of the viral sequences between the cleavage sites recognized by the CRISPR endonuclease. The excised region can vary in size from a single nucleotide to several thousand nucleotides. Exemplary excised regions are described herein.

When the compositions are administered as a nucleic acid or are contained within an expression vector, the CRISPR endonuclease can be encoded by the same nucleic acid or vector as the guide RNA sequences. Alternatively, or in addition, the CRISPR endonuclease can be encoded in a physically separate nucleic acid from the guide RNA sequences or in a separate vector.

In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-di methylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl) uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶N-methyladenosine; N^{6,}N⁶-dimethyladenosine; N⁶,2′-O-trimethyladenosine; 2-methylthio-N⁶N-isopentenyladenosine; N⁶-(cis-hydroxyisopentenyl)-adenosine; 2-methylthio-N⁶-(cis-hydroxyisopentenyl)-adenosine; N⁶-glycinylcarbamoyl) adenosine; N⁶-threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1;2′-O-dimethyl inosine; 2′-O-methyl guanosine; 1-methyl guanosine; N²-methyl guanosine; N^{2,}N²-dimethyl guanosine; N²,2′-O-dimethyl guanosine; N^{2,}N^{2,2′}-O-trimethyl guanosine; 2′-O-ribosyl guanosine (phosphate); 7-methyl guanosine; N²;7-dimethyl guanosine; N²;N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

In certain embodiments, the composition comprises multiple different gRNAs, each targeted to a different target sequence. In certain embodiments, this multiplexed strategy provides for increased efficacy. In some embodiments, the compositions described herein utilize about 1 gRNA to about 6 gRNAs. In some embodiments, the compositions described herein utilize at least about 1 gRNA. In some embodiments, the compositions described herein utilize at most about 6 gRNAs. In some embodiments, the compositions described herein utilize about 1 gRNA to about 2 gRNAs, about 1 gRNA to about 3 gRNAs, about 1 gRNA to about 4 gRNAs, about 1 gRNA to about 5 gRNAs, about 1 gRNA to about 6 gRNAs, about 2 gRNAs to about 3 gRNAs, about 2 gRNAs to about 4 gRNAs, about 2 gRNAs to about 5 gRNAs, about 2 gRNAs to about 6 gRNAs, about 3 gRNAs to about 4 gRNAs, about 3 gRNAs to about 5 gRNAs, about 3 gRNAs to about 6 gRNAs, about 4 gRNAs to about 5 gRNAs, about 4 gRNAs to about 6 gRNAs, or about 5 gRNAs to about 6 gRNAs. In some embodiments, the compositions described herein utilize about 1 gRNA, about 2 gRNAs, about 3 gRNAs, about 4 gRNAs, about 5 gRNAs, or about 6 gRNAs.

In some embodiments, the gRNA is a synthetic oligonucleotide. In some embodiments, the synthetic nucleotide comprises a modified nucleotide. Modification of the inter-nucleoside linker (i.e. backbone) can be utilized to increase stability or pharmacodynamic properties. For example, inter-nucleoside linker modifications prevent or reduce degradation by cellular nucleases, thus increasing the pharmacokinetics and bioavailability of the gRNA. Generally, a modified inter-nucleoside linker includes any linker other than other than phosphodiester (PO) liners, that covalently couples two nucleosides together. In some embodiments, the modified inter-nucleoside linker increases the nuclease resistance of the gRNA compared to a phosphodiester linker. For naturally occurring oligonucleotides, the inter-nucleoside linker includes phosphate groups creating a phosphodiester bond between adjacent nucleosides. In some embodiments, the gRNA comprises one or more inter-nucleoside linkers modified from the natural phosphodiester. In some embodiments all of the inter-nucleoside linkers of the gRNA, or contiguous nucleotide sequence thereof, are modified. For example, in some embodiments the inter-nucleoside linkage comprises sulfur(S), such as a phosphorothioate inter-nucleoside linkage.

Modifications to the ribose sugar or nucleobase can also be utilized herein. Generally, a modified nucleoside includes the introduction of one or more modifications of the sugar moiety or the nucleobase moiety. In some embodiments, the gRNAs, as described, comprise one or more nucleosides comprising a modified sugar moiety, wherein the modified sugar moiety is a modification of the sugar moiety when compared to the ribose sugar moiety found in deoxyribose nucleic acid (DNA) and RNA. Numerous nucleosides with modification of the ribose sugar moiety can be utilized, primarily with the aim of improving certain properties of oligonucleotides, such as affinity and/or stability. Such modifications include those where the ribose ring structure is modified. These modifications include replacement with a hexose ring (HNA), a bicyclic ring having a biradical bridge between the C2 and C4 carbons on the ribose ring (e.g. locked nucleic acids (LNA)), or an unlinked ribose ring which typically lacks a bond between the C2 and C3 carbons (e.g. UNA). Other sugar modified nucleosides include, for example, bicyclohexose nucleic acids or tricyclic nucleic acids. Modified nucleosides also include nucleosides where the sugar moiety is replaced with a non-sugar moiety, for example in the case of peptide nucleic acids (PNA), or morpholino nucleic acids.

Sugar modifications also include modifications made by altering the substituent groups on the ribose ring to groups other than hydrogen, or the 2′-OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity. A 2′ sugar modified nucleoside is a nucleoside that has a substituent other than H or-OH at the 2′ position (2′ substituted nucleoside) or comprises a 2′ linked biradicle, and includes 2′ substituted nucleosides and LNA (2′-4′ biradicle bridged) nucleosides. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. By way of further example, in some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the gRNA comprises one or more modified sugars. In some embodiments, the gRNA comprises only modified sugars. In certain embodiments, the gRNA comprises greater than 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the gRNA comprises both inter-nucleoside linker modifications and nucleoside modifications.

Target specificity can be used in reference to a guide RNA, or a crRNA specific to a target polynucleotide sequence or region (e.g., LTR nucleic acid sequences, Gag nucleic acid sequences, Pol nucleic acid sequences, Pro nucleic acid sequences, Env nucleic acid sequences, MOGS gene sequences or combinations thereof) and further includes a sequence of nucleotides capable of selectively annealing/hybridizing to a target (sequence or region) of a target polynucleotide (e.g. corresponding to a target), e.g., a target DNA. In some embodiments, a crRNA or the derivative thereof contains a target-specific nucleotide region complementary to a region of the target DNA sequence. In some embodiments, a crRNA or the derivative thereof contains other nucleotide sequences besides a target-specific nucleotide region. In some embodiments, the other nucleotide sequences are from a tracrRNA sequence.

gRNAs are generally supported by a scaffold, wherein a scaffold refers to the portions of gRNA or crRNA molecules comprising sequences which are substantially identical or are highly conserved across natural biological species (e.g. not conferring target specificity). Scaffolds include the tracrRNA segment and the portion of the crRNA segment other than the polynucleotide-targeting guide sequence at or near the 5′ end of the crRNA segment, excluding any unnatural portions comprising sequences not conserved in native crRNAs and tracrRNAs. In some embodiments, the crRNA or tracrRNA comprises a modified sequence. In certain embodiments, the crRNA or tracrRNA comprises at least 1, 2, 3, 4, 5, 10, or 15 modified bases (e.g. a modified native base sequence).

Complementary, as used herein, generally refers to a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A: T, A: U, and G: C, by Watson-Crick and Hoogsteen-type hydrogen bonding. In some embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application and can be routinely determined by persons skilled in the art, without undue experimentation. Hybridization generally refers to process in which two single-stranded polynucleotides bind non-covalently to form a stable double-stranded polynucleotide. A resulting double-stranded polynucleotide is a “hybrid” or “duplex.” In certain instances, 100% sequence identity is not required for hybridization and, in certain embodiments, hybridization occurs at about greater than 70%, 75%, 80%, 85%, 90%, or 95% sequence identity. In certain embodiments, sequence identity includes in addition to non-identical nucleobases, sequences comprising insertions and/or deletions.

The nucleic acid of the disclosure, including the RNA (e.g., crRNA, tracrRNA, gRNA) or nucleic acids encoding the RNA, may be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, 2^{nd} edition, Dieffenbach and Dveksler, eds., Cold Spring Harbor Laboratory Press, 2003. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

The isolated nucleic acids also can be chemically synthesized, either as a single nucleic acid (e.g., using automated DNA synthesis in the 3′ to 5′ direction using phosphoramidite technology) or as a series of oligonucleotides. Isolated nucleic acids of the disclosure also can be obtained by mutagenesis of, e.g., a naturally occurring portion crRNA, tracrRNA, RNA-encoding DNA, or of a Cas9-encoding DNA

In certain embodiments, the isolated RNA are synthesized from an expression vector encoding the RNA molecule, as described in detail elsewhere herein.

### Pharmaceutical Compositions

The compositions described herein are suitable for use in a variety of drug delivery systems described above. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue-specific antibody. The liposomes will be targeted to and taken up selectively by the organ.

The present disclosure also provides pharmaceutical compositions comprising one or more of the compositions described herein. Formulations may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

Administration of the compositions of this disclosure may be carried out, for example, by parenteral, by intravenous, intratumoral, subcutaneous, intramuscular, or intraperitoneal injection, or by infusion or by any other acceptable systemic method. Formulations for administration of the compositions include those suitable for rectal, nasal, oral, topical (including buccal and sublingual), vaginal or parenteral (including subcutaneous, intramuscular, intravenous and intradermal) administration. The formulations may conveniently be presented in unit dosage form, e.g. tablets and sustained release capsules, and may be prepared by any methods well known in the art of pharmacy.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the disclosure are known in the art and described, for example in Genaro, ed. (1985, Remington's Pharmaceutical Sciences, Mack Publishing Co., Easton, PA), which is incorporated herein by reference.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

In an embodiment, the composition includes an anti-oxidant and a chelating agent that inhibits the degradation of one or more components of the composition. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension the composition of the disclosure in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

### Combination Therapies

In certain embodiments, the gene-editing compositions embodied herein are administered to a patient in combination with one or more other anti-viral agents or therapeutics. The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

Examples include any molecules that are used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof.

Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs. The therapeutic agents may be administered under a metronomic regimen. As used herein, “metronomic” therapy refers to the administration of continuous low-doses of a therapeutic agent.

The compositions can be administered in conjunction with (e.g., before, simultaneously or following) one or more therapies. For example, in certain embodiments, the method comprises administration of a composition of the disclosure in conjunction with an additional anti-viral therapy, cancer therapy and the like.

### Methods of Treatment

The present disclosure provides a method of treating or preventing a retrovirus, e.g., HIV infection. In some embodiments, the method comprises administering to a subject in need thereof, an effective amount of a composition comprising at least one of a guide nucleic acid and a Cas peptide, or functional fragment or derivative thereof. In some embodiments, the method comprises administering a composition comprising an isolated nucleic acid encoding at least one of: the guide nucleic acid and a Cas peptide, or functional fragment or derivative thereof. In certain embodiments, the method comprises administering a composition described herein to a subject diagnosed with a retrovirus, e.g., HIV infection, at risk for developing a retrovirus, e.g., HIV infection and the like.

Dosage, toxicity and therapeutic efficacy of the present compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. The Cas9/gRNA compositions that exhibit high therapeutic indices are preferred. While Cas9/gRNA compositions that exhibit toxic side effects may be used, care should be taken to design a delivery system that targets such compositions to the site of affected tissue in order to minimize potential damage to uninfected cells and, thereby, reduce side effects.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the disclosure, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a composition (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the disclosure can include a single treatment or a series of treatments.

The gRNA expression cassette can be delivered to a subject by methods known in the art. In some aspects, the Cas may be a fragment wherein the active domains of the Cas molecule are included, thereby cutting down on the size of the molecule. Thus, the, Cas/gRNA molecules can be used clinically, similar to the approaches taken by current gene therapy. In some embodiments, the method comprises genetically modifying a cell to express a guide nucleic acid and/or Cas peptide. For example, in some embodiments, the method comprises contacting a cell with an isolated nucleic acid encoding the guide nucleic acid and/or Cas peptide.

In some embodiments, for viral vector-mediated delivery, a dose comprises at least 1×10⁵ particles to about 1×10¹⁵ particles. In some embodiments the delivery is via an adenovirus, such as a single dose containing at least 1×10⁵ particles (also referred to as particle units, pu) of adenoviral vector. In some embodiments, the dose is at least about 1×10⁶ particles (for example, about 1×10⁶-1×10¹² particles), at least about 1×10⁷ particles, at least about 1×10⁸ particles (e.g., about 1×10⁸-1×10¹¹ particles or about 1×10⁸-1×10¹² particles), at least about 1×10⁰ particles (e.g., about 1×10⁹-1×10¹⁰ particles or about 1×10⁹-1×10¹² particles), or at least about 1×10¹⁰ particles (e.g., about 1×10⁻1×10¹² particles) of the adenoviral vector. Alternatively, the dose comprises no more than about 1×10¹⁴ particles, no more than about 1×10¹³ particles, no more than about 1 ×10¹² particles, no more than about 1×10¹¹ particles, and no more than about 1×10¹⁰ particles (e.g., no more than about 1×10⁹ particles). Thus, in some embodiments, the dose contains a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1× 108 pu, about 2×10⁸ pu, about 4×10⁸ pu, about 1×10⁹ pu, about 2×10⁹ pu, about 4×10⁹ pu, about 1× 1010 pu, about 2×10¹⁰ pu, about 4×10¹⁰ pu, about 1× 1011 pu, about 2×10¹ pu, about 4×10¹¹ pu, about 1×10¹² pu, about 2×10¹² pu, or about 4×10¹² pu of adenoviral vector. In some embodiments, the adenovirus is delivered via multiple doses.

In some embodiments, the delivery is via an AAV. A therapeutically effective dosage for in vivo delivery of the AAV to a human is believed to be in the range of from about 20 to about 50 ml of saline solution containing from about 1×10¹⁰ to about 1×10¹⁰ functional AAV/ml solution. The dosage can be adjusted to balance therapeutic benefit against any side effects. In some embodiments, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. In some embodiments, a human dosage is about 1×10¹³ genomes AAV. In some embodiments, such concentrations are delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves (see, for example, U.S. Pat. No. 8,404,658).

In some embodiments, the cell is genetically modified in vivo in the subject in whom the therapy is intended. In certain aspects, for in vivo, delivery the nucleic acid is injected directly into the subject. For example, in some embodiments, the nucleic acid is delivered at the site where the composition is required. In vivo nucleic acid transfer techniques include, but is not limited to, transfection with viral vectors such as adenovirus, Herpes simplex I virus, adeno-associated virus), lipid-based systems (useful lipids for lipid-mediated transfer of the gene are DOTMA, DOPE and DC-Chol, for example), naked DNA, and transposon-based expression systems. Exemplary gene therapy protocols see Anderson et al., Science 256:808-813 (1992). See also WO 93/25673 and the references cited therein. In certain embodiments, the method comprises administering of RNA, for example mRNA, directly into the subject (see for example, Zangi et al., 2013 Nature Biotechnology, 31:898-907).

For ex vivo treatment, an isolated cell is modified in an ex vivo or in vitro environment. In some embodiments, the cell is autologous to a subject to whom the therapy is intended. Alternatively, the cell can be allogeneic, syngeneic, or xenogeneic with respect to the subject. The modified cells may then be administered to the subject directly.

One skilled in the art recognizes that different methods of delivery may be utilized to administer an isolated nucleic acid into a cell. Examples include: (1) methods utilizing physical means, such as electroporation (electricity), a gene gun (physical force) or applying large volumes of a liquid (pressure); and (2) methods wherein the nucleic acid or vector is complexed to another entity, such as a liposome, aggregated protein or transporter molecule.

The amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present disclosure (for instance, the cost associated with synthesis). One skilled in the art can easily make any necessary adjustments in accordance with the exigencies of the particular situation.

Genetically modified cells may also contain a suicide gene i.e., a gene which encodes a product that can be used to destroy the cell. In many gene therapy situations, it is desirable to be able to express a gene for therapeutic purposes in a host, cell but also to have the capacity to destroy the host cell at will. The therapeutic agent can be linked to a suicide gene, whose expression is not activated in the absence of an activator compound. When death of the cell in which both the agent and the suicide gene have been introduced is desired, the activator compound is administered to the cell thereby activating expression of the suicide gene and killing the cell. Examples of suicide gene/prodrug combinations which may be used are herpes simplex virus-thymidine kinase (HSV-tk) and ganciclovir, acyclovir; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

### EXAMPLES

### Example 1

### CRISPR Induced Disruption of MOGS Gene Inhibits HIV Progeny Virion Infectivity

Using CRISPR-Cas9, the effects of MOGS gene knockout were tested on HIV-1 infection. First, we developed and characterized single-cell MOGS gene knockout clones in TZM-bl and Jurkat cells. Next, cells were challenged by infection with the HIV-1 NL4-3-GFP reporter virus. No significant differences were detected in the level of primary infection in MOGS−/− cells comparing to WT cells, as measured by GFP reporter positivity and Gag p24 levels in supernatants from infected cells. However, virions released from infected MOGS negative cells showed markedly reduced of infectivity, which was examined by secondary infections of highly susceptible to HIV-1, HutR5 T-lymphoid cell line. Western blot analysis confirmed a lack of proper glycan trimming of gp160 precursor protein in HIV-1 infected MOGS^{-/-} cells. Presented CRISPR based disruption of the MOGS gene provides a new, resistance-refractory antiviral strategy to block HIV-1 infection.

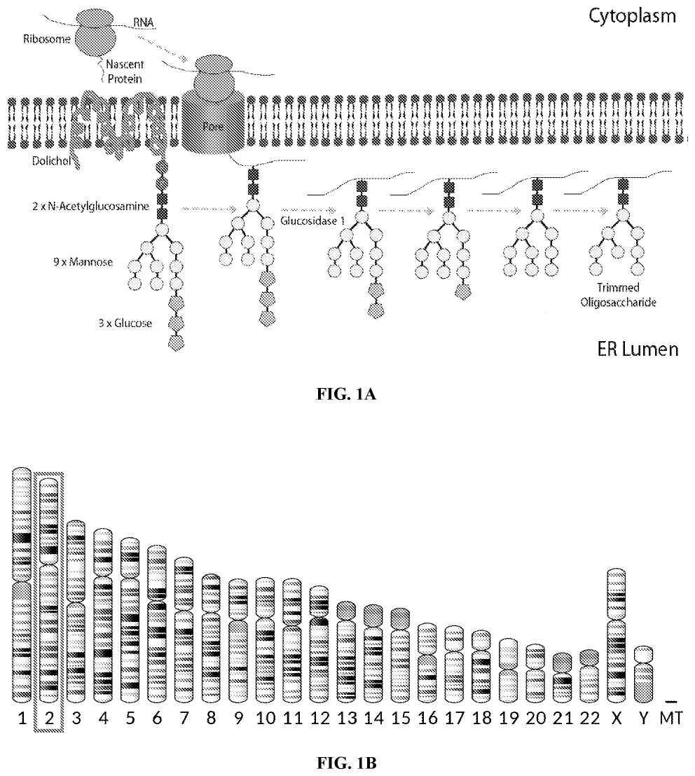

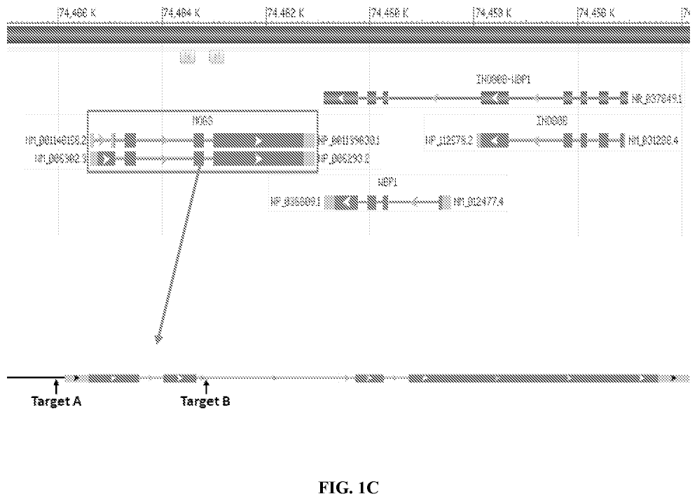

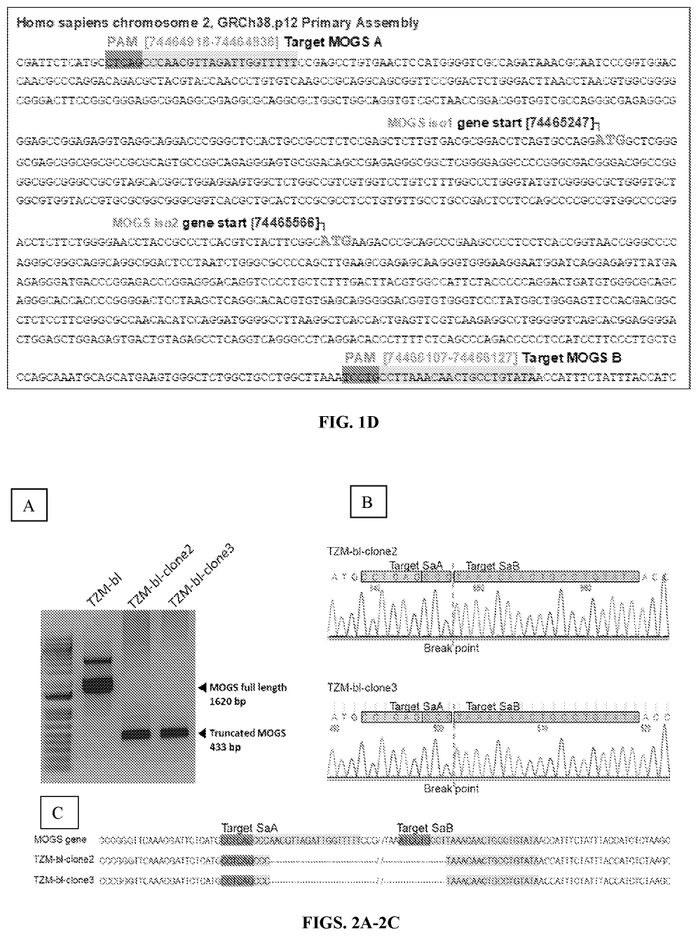

Mannosyl-oligosaccharide glucosidase (MOGS, GC-1) is a transmembrane endoplasmic reticulum (ER) enzyme responsible for the initial step of remodeling of the N-linked glycans, i.e., removal of the first glucose residue, what allows further trimming and maturation of glycoproteins (FIG. 1A). The human MOGS gene is located on chromosome 2 (2p13.1); it is 4326 bp long and has a total of 4 exons (FIGS. 1B, 1C). A pair of guide RNAs were designed targeting the promoter and intron2 regions of the human MOGS gene. Successful cleavage at the target sites lead to the deletion of the 1187bp long segment of DNA spanning exon1-intron1-exon2 and containing the MOGS start codons (FIG. 1D) resulting in the knockout of MOGS expression.

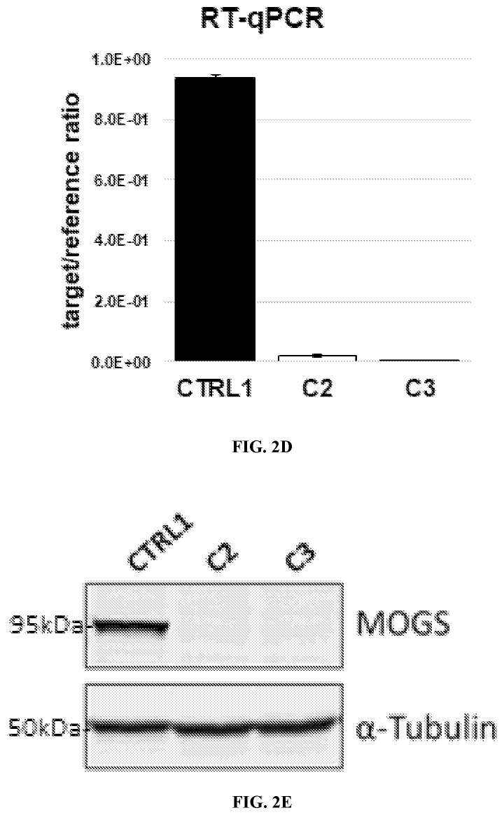

In FIGS. 2A-2E, TZM-bl cells were transfected with in pX601-no-gRNA (control) or pX601-MOGS-A/MOGS-B plasmid together with pKLV-BFP (to provide selection marker) at ratio 10:1, then were selected for two weeks with 1 μg/ml puromycin and clonally expanded. Genomic DNA was extracted from one control and two MOGS knockout single-cell clones and subjected to PCRs specific to the exon1-intron1-exon2 region of the MOGS gene. Gel agarose electrophoresis confirmed the presence of CRISPR-Cas9 induced, double-cleaved/end-joined truncated amplicons in pX601-MOGS-A/MOGS-B treated clones (FIG. 2A). Truncated PCR products were verified by Sanger sequencing shown in (FIG. 2B). Representative alignment of the sequencing results from TZM-bl single-cell clones (FIG. 2C) gRNAs target sequences are highlighted in green, PAMs in red. Lack of MOGS expression was in knockout single-cell clones was confirmed or mRNA and protein level by RT-PCR (FIG. 2D) and Western blot (FIG. 2E), respectively.

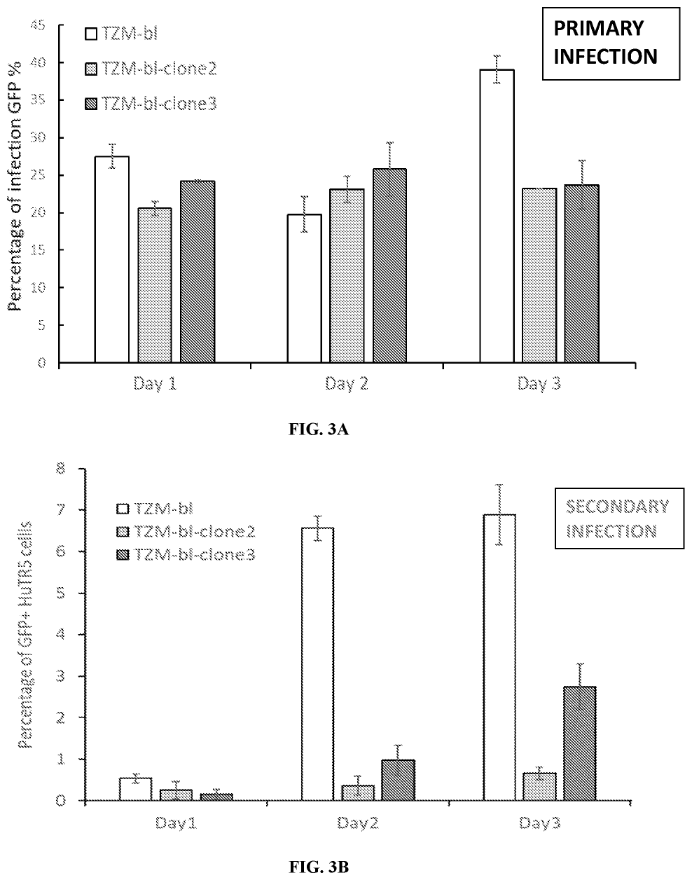

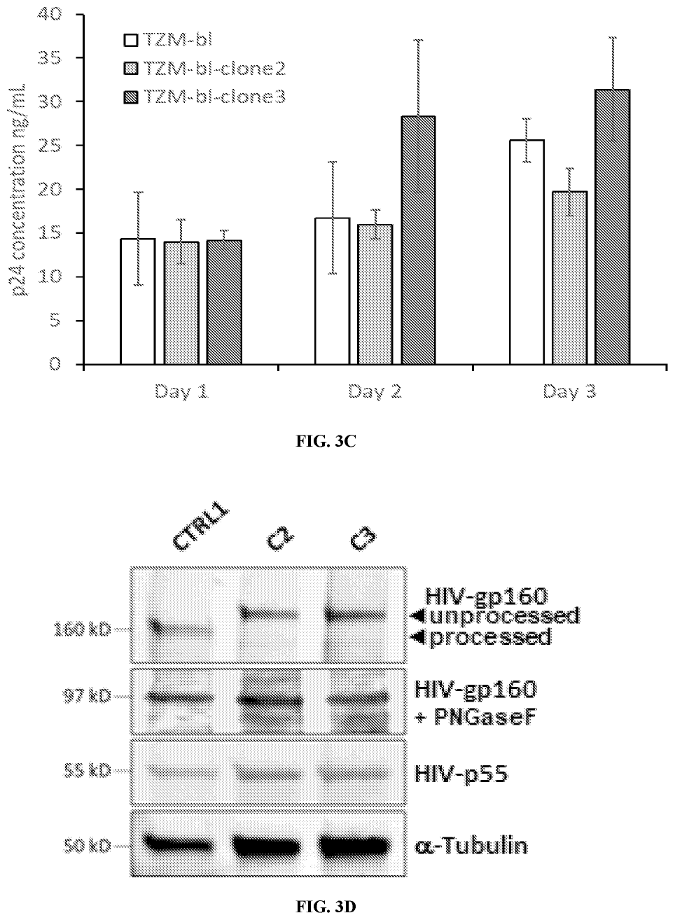

In the experiments conducted and the results shown in FIGS. 3A-3D, one WT and two MOGS knockout (clone2 and clone 3) TZM-bl single-cell clones were infected with CCR5-tropic HIV-1NL4-3-BAL-GFP reporter virus at MOI 0.25 (FIG. 3A). Supernatants from infected cells were collected every day for a total of 3 days and tested for infectivity by incubation with highly susceptible to HIV infection, HuTR5 cell line. After 48 h incubation, HutR5 cells were fixed with paraformaldehyde and analyzed for GFP expression by flow cytometry (FIG. 3B). The levels of the virus in supernatants used for secondary infection experiment were quantified by HIV Gag p24 ELISA (FIG. 3C) Additionally, at 48 h time point, some of the TZM-bl cells were harvested, and protein lysates were examined for HIV gp160 and p55 proteins expression by Western Blot (FIG. 3D).

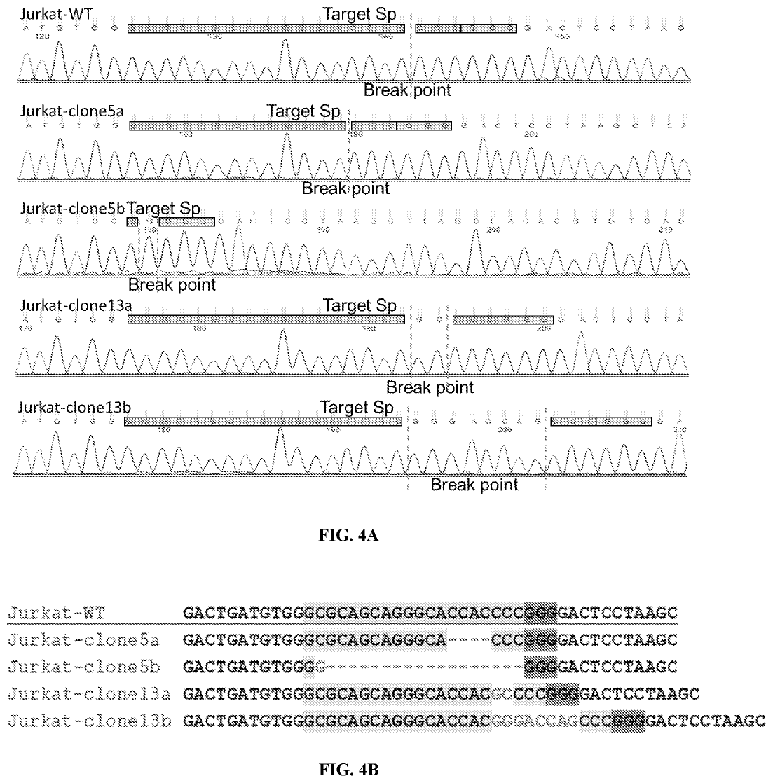

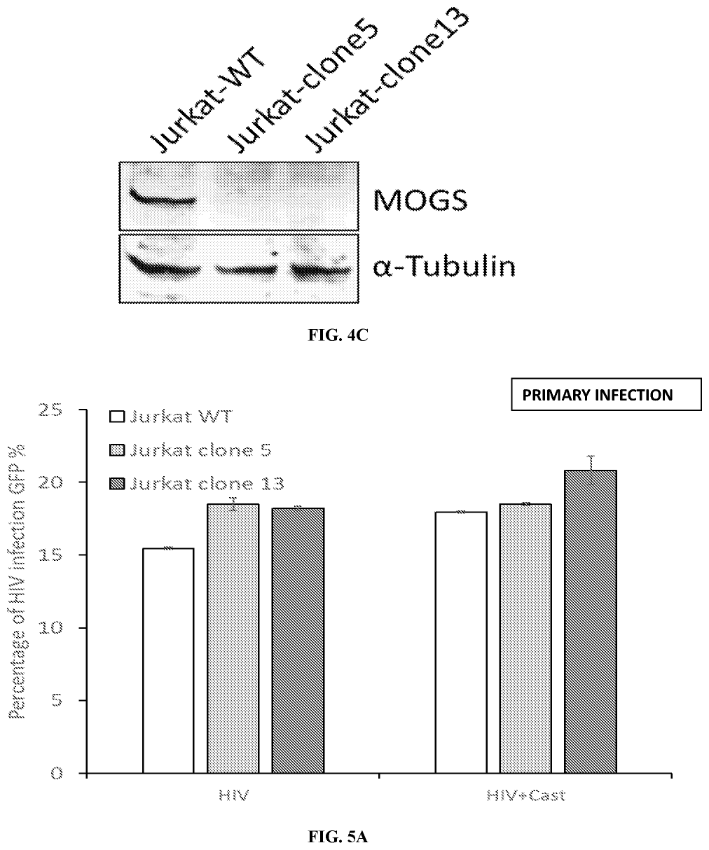

Jurkat cells were electroporated with synthetic gRNA targeting exon2 of MOGS gene and recombinant Cas9 protein (SYNTHEGO) and then clonally expanded and screened for the MOGS gene expression by Western blot. One WT (control) and two MOGS knockout (clone 5 and 13) single-cell clones were selected for further studies. In FIG. 4A, Sanger sequencing results are shown, verifying the presence of InDel mutations at the gRNA target sites at both genomic loci (complete biallelic knockout). Sequences alignment in FIG. 4B. In FIG. 4C, Western blot conducted, verified the Lack of MOGS expression in knockout clones.

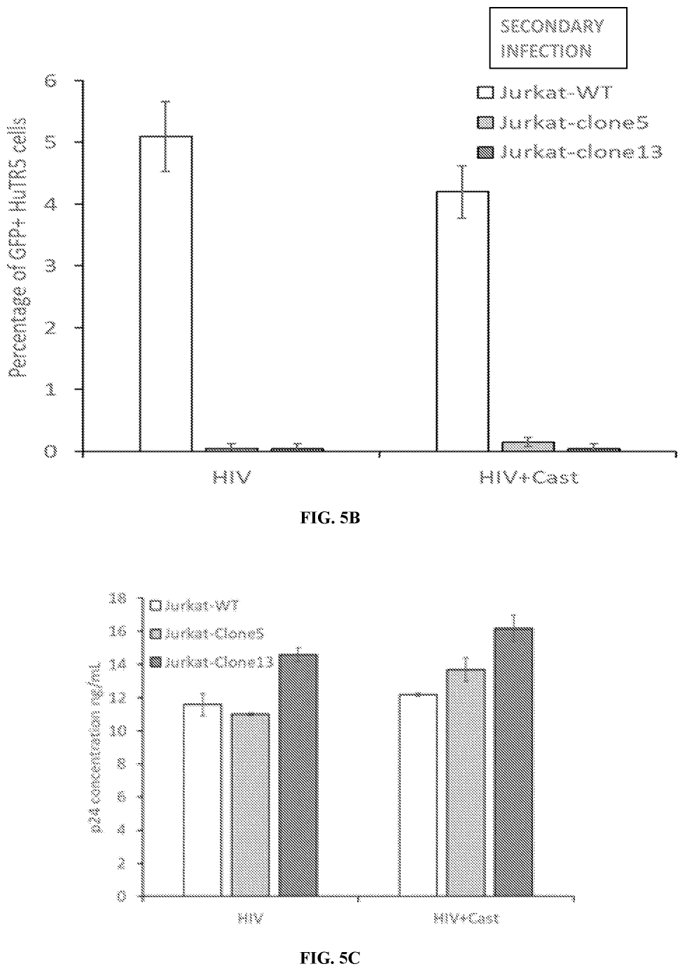

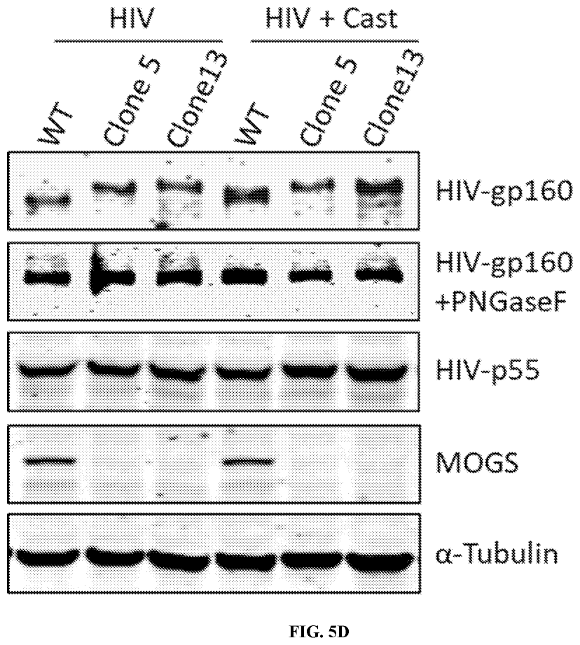

One WT and two MOGS knockout (clone5 and clone 13) Jurkat single-cell clones were infected with CCR5-tropic HIV-1NL4-3-BAL-GFP reporter virus at MOI 0.25 (FIG. 5A.). After 48 h, supernatants from infected cells were collected and tested for infectivity by incubation with highly susceptible to HIV infection, HuTR5 cell line. After 48 h incubation, HutR5 cells were fixed with paraformaldehyde and analyzed for GFP expression by flow cytometry (FIG. 5B). The levels of the virus in supernatants used for secondary infection experiment were quantified by HIV Gag p24 ELISA (FIG. 5C.) Additionally, Jurkat cells were harvested, and protein lysates were examined for HIV gp160, p55, and MOGS proteins expression by Western Blot (FIG. 5D). Glycosidase inhibitor, castanospermine, used at high 10 μg/ml concentration, showed very modest effects on progeny virus infectivity (FIG. 5B) and processing of gp160 (FIG. 5D).

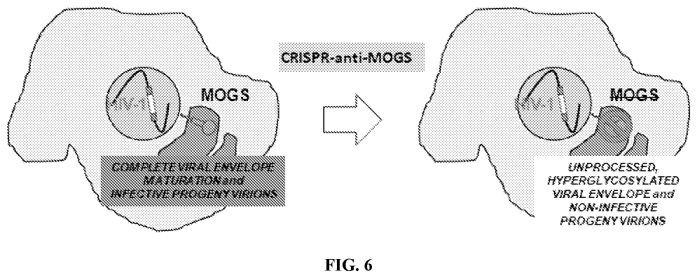

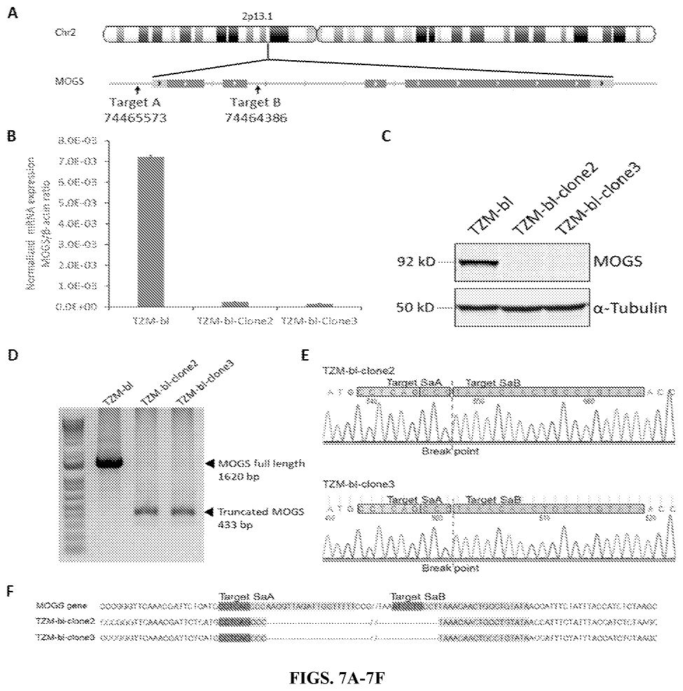

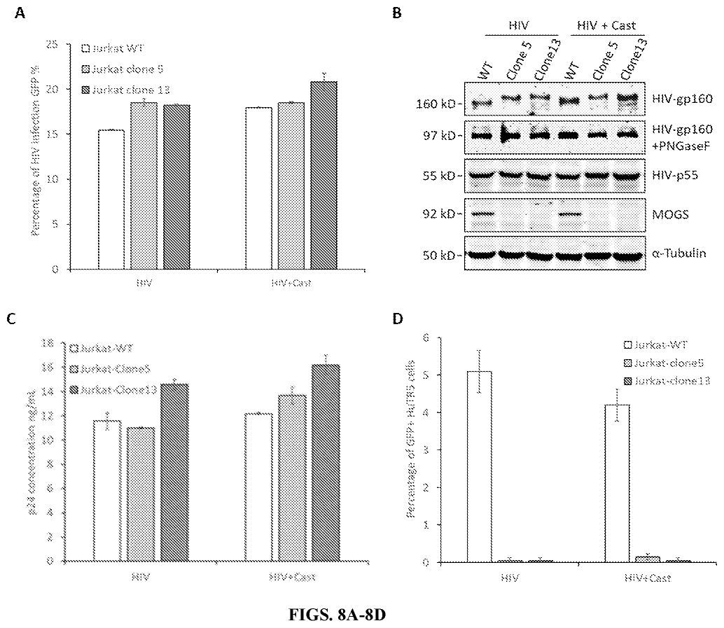

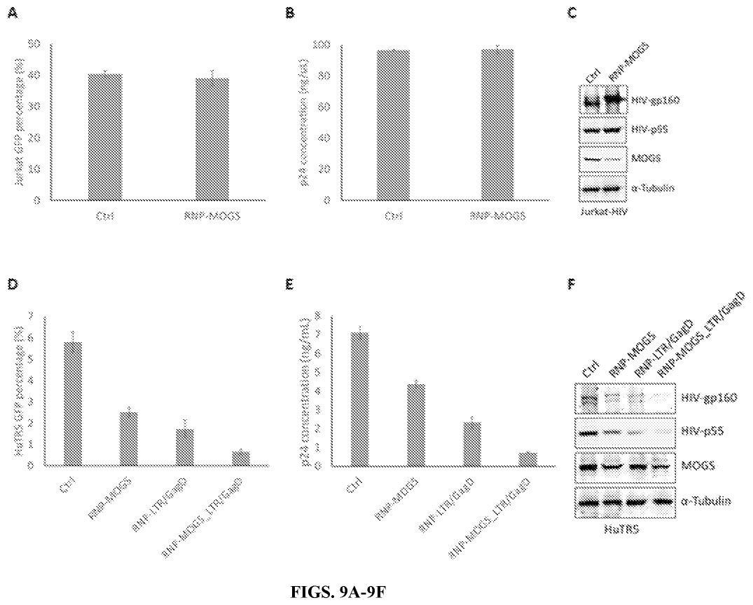

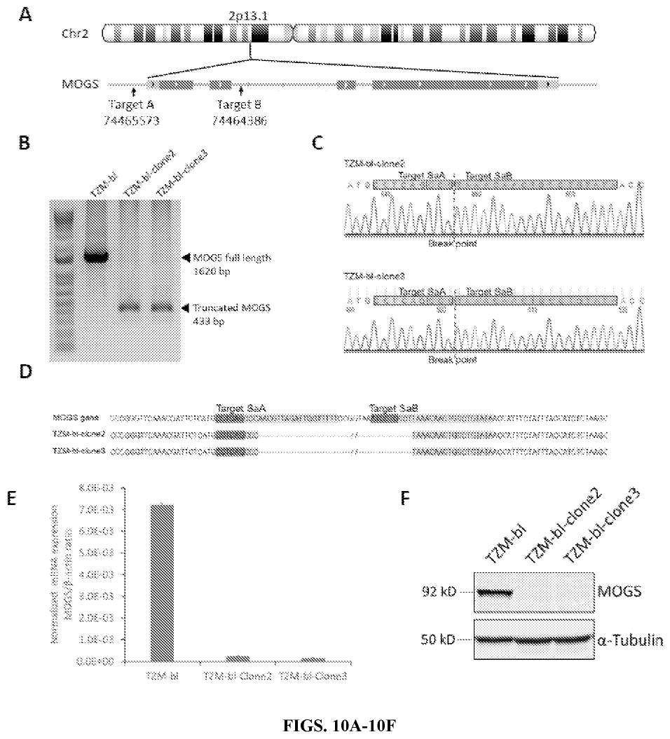

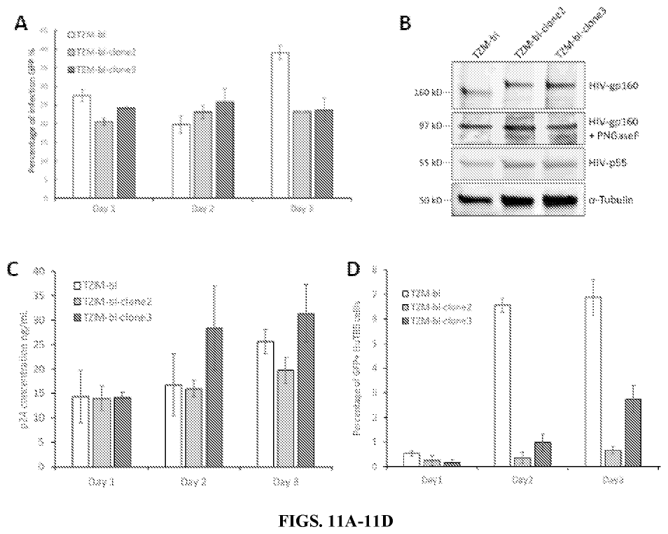

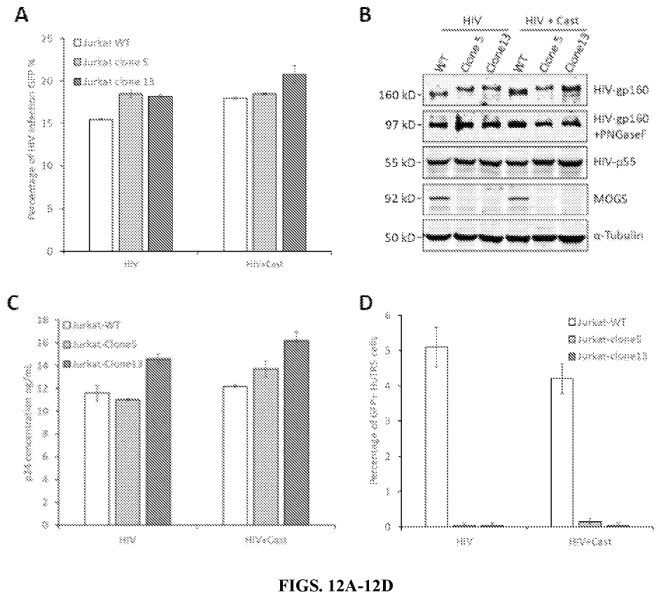

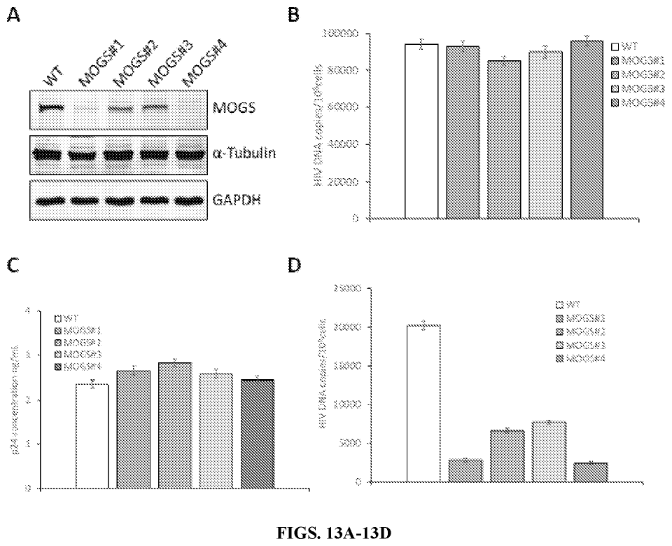

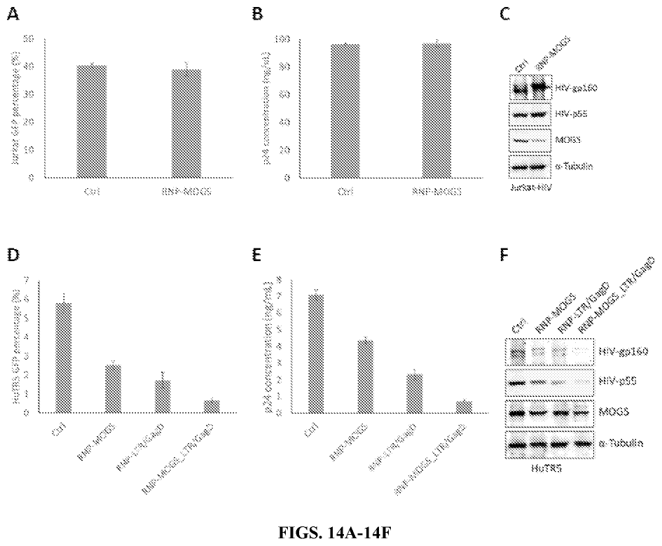

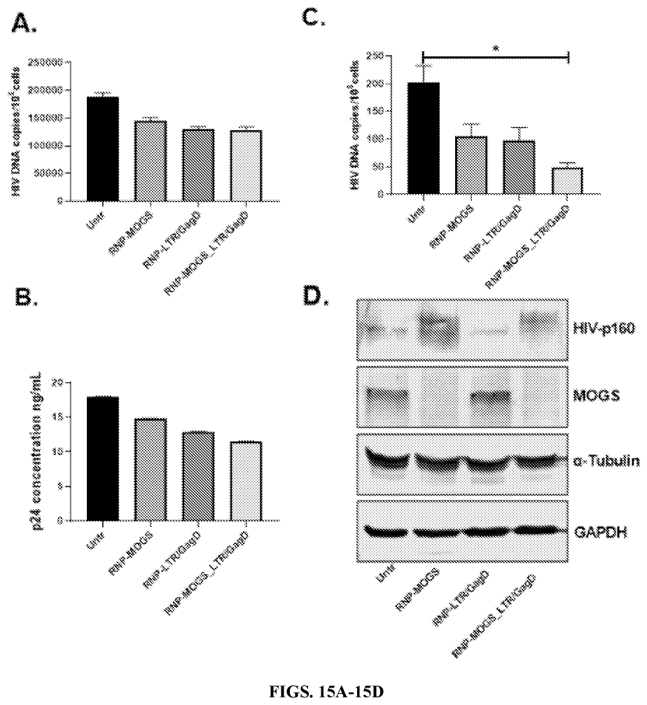

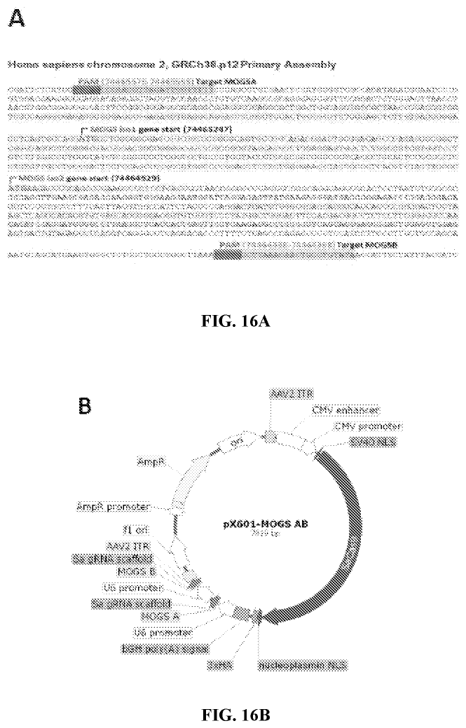

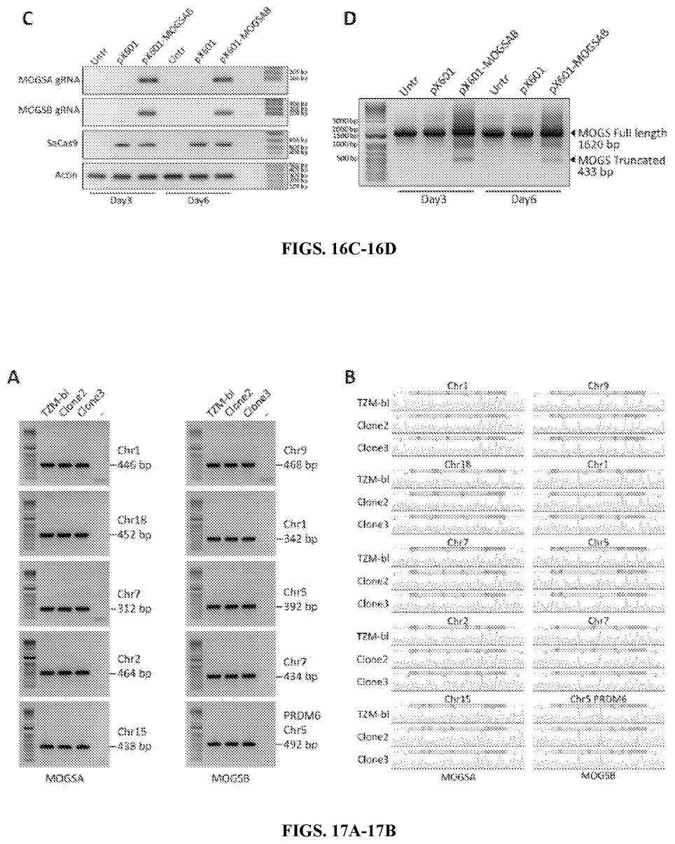

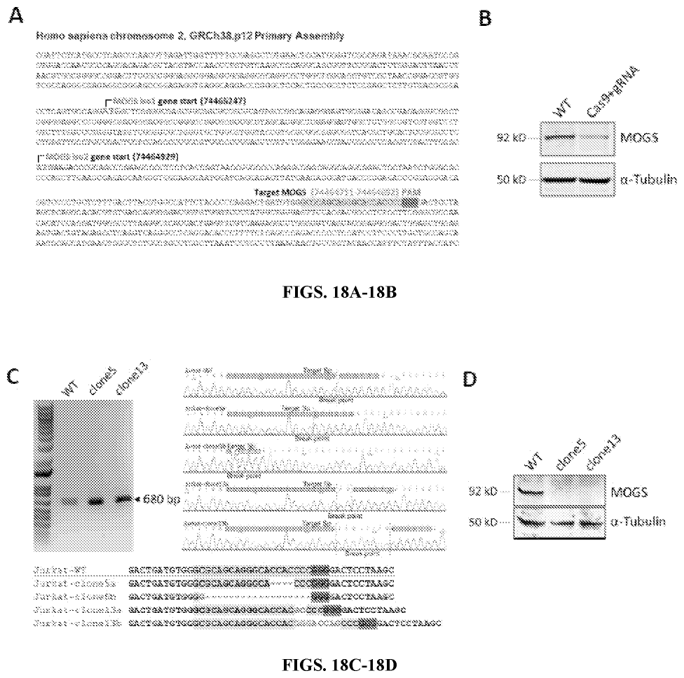

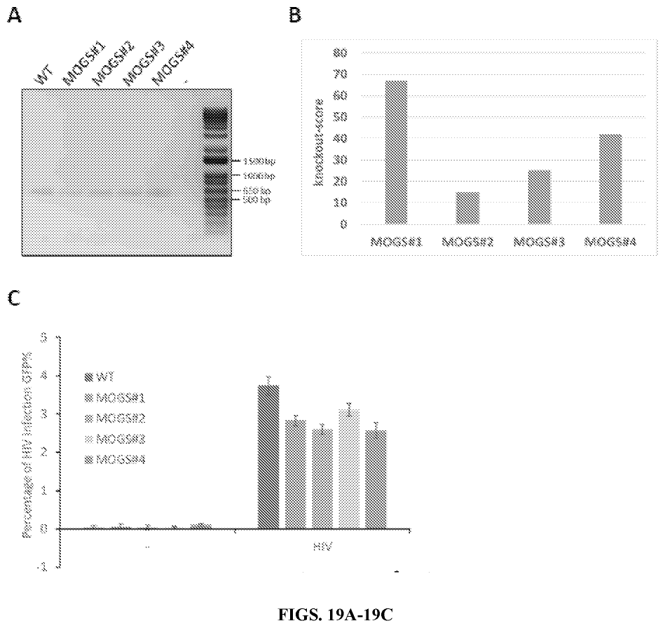

FIG. 6 is a schematic representation showing CRISPR-Cas9 mediated knockout of MOGS gene expression in the host cells has potent antiviral activity. Lack of proper remodeling of glycan chains, decorating the viral envelope, results in non-infective progeny virions and breaks the infection cycle of the HIV-1.

TABLE 1
Potential off target sites in human genome for MOGS target A and B.
Table discloses SEQ ID NOS 27-75, 75-85, 84, and 86-126, respectively,
in order of appearance.

| MOGSA sequence | PAM | On-target Score | Off-target Score |
|---|---|---|---|
| AAAAACCAATCTAACGTTGGG | CTGAG | 22.9 | 93.5 |

| Sequence | PAM | Score | Gene | chromosome | Mismatch | On-target |
|---|---|---|---|---|---|---|
| AATAACCAATATAACGTTTGG | AGGGA | 0.5 | | chr1:−223007178 | 3 | FALSE |
| TAAAACTCATCTAAAGTTGGG | CAGAG | 0.4 | | chr18:+75408839 | 4 | FALSE |
| TTAAACCAACCTAAAGTTGGC | AGGGA | 0.1 | | chr7:−145084841 | 5 | FALSE |
| TCAAATCAATCTAACTTTTGG | ATGGA | 0.1 | | chr2:−1252277 | 5 | FALSE |
| AAACAGCAATCCAACGTTGGA | CAGAA | 0.1 | | chr15:+89058842 | 4 | FALSE |
| ATAAACCAATGTAAAGTTTGG | GGGGA | 0.1 | | chr18:−77125281 | 4 | FALSE |
| TAAAATAAATCTAACGTTTTG | GAGAA | 0.1 | | chr7:−39007024 | 5 | FALSE |
| ATAAACCAATCTTACTTTAGG | AAGGA | 0.1 | | chrY:−6848825 | 4 | FALSE |
| AAAAATAAATCTAAAGTTGGA | GAGGG | 0.1 | | chr5:−59064772 | 4 | FALSE |
| TAAAATGAATCTAACTTTGAG | TGGAA | 0.1 | | chr4:−168055825 | 5 | FALSE |
| TAAAAGCAATCTTACGTTTGT | TTGAA | 0.1 | | chr12:−45151581 | 5 | FALSE |
| TAATACCAATTTAACGGAGGG | TAGGG | 0.1 | | chr3:−182494891 | 5 | FALSE |
| CAAAATCAATCTAATGTAGGA | AAGGA | 0.1 | | chr2:−141710926 | 5 | FALSE |
| ATAATCCACTCTAACTTTGGG | CAGGA | 0.1 | | chr11:+4179686 | 4 | FALSE |
| TAAAACTAATATAAAGTTGGA | AAGAA | 0.1 | | chr3:−82582701 | 5 | FALSE |
| GAAAACAAATATAAGGTTGGA | TTGGG | 0.1 | | chr5:+71583444 | 5 | FALSE |
| ATTAACCAGTCTAAGGTTGGG | ATGGA | 0.1 | | chr1:−83708474 | 4 | FALSE |
| AAAAACCTATGTAACGTTTAG | GGGAA | 0.1 | | chr1:−164679124 | 4 | FALSE |
| GAAAACAAATCTAACCTTATG | TGGAA | 0.1 | | chr9:−113329284 | 5 | FALSE |
| AAAAACTAATTTAACATTAGG | TAGAA | 0.1 | | chr10:+16939791 | 4 | FALSE |
| AAAAATCCATCTAATGGTGGG | TGGGA | 0.1 | | chr4:−181429293 | 4 | FALSE |
| AAATAACAAGCTAACTTTGGG | AGGAG | 0.1 | | chr3:−174640046 | 4 | FALSE |
| TCAGGCCAATCTAATGTTGGG | TGGAA | 0.1 | | chr1:−209342435 | 5 | FALSE |
| TAACATCAATCTGAAGTTGGG | GTGAG | 0.1 | | chr20:+20306629 | 5 | FALSE |
| CAAGACCAATGTATCATTGGG | AGGAG | 0.1 | | chr6:−11666409 | 5 | FALSE |
| AAATCCCAAGCTAAAGTTGGG | AGGAG | 0.1 | | chr6:+99270525 | 4 | FALSE |
| CAAAACCCATCTACCCTTGCG | CGGGG | 0.1 | | chr17:−45161763 | 5 | FALSE |
| CAAAACCAAAATAACATTGGA | AGGAA | 0.1 | | chr4:+138853014 | 5 | FALSE |
| CACAACCATTATAAAGTTGGG | CTGAG | 0.1 | | chr3:−96829074 | 5 | FALSE |
| GAAAACAAATCTAAAATTTGG | CAGAA | 0.1 | | chr2:−227631204 | 5 | FALSE |
| AAAAACCCATCTGCCGTTGAG | AGGAA | 0.1 | | chr20:+55451376 | 4 | FALSE |
| GAAAATCAATCTGATGGTGGG | TCGGG | 0.1 | | chr7:+45709300 | 5 | FALSE |
| TAAAACCAACCTCAAGTTGGT | GGGGA | 0.1 | | chr6:−35202448 | 5 | FALSE |
| CAAAACCAACCTAACTTGGGT | TTGAG | 0.1 | | chr4:−169047258 | 5 | FALSE |
| CAACACCAAACCAAAGTTGGG | TTGAA | 0.1 | | chr2:+229585812 | 5 | FALSE |
| GAAATCCAAGGTAACCTTGGG | CTGAG | 0.1 | | chr8:−57256433 | 5 | FALSE |
| TAAAAGCAATGTAATATTGGG | AGGGG | 0.1 | | chr4:+165706353 | 5 | FALSE |
| AAAAAAGAATTTAACTTTGGG | TAGAA | 0.1 | | chr4:+74769673 | 4 | FALSE |
| AAAAAGTAATATAACATTGGG | ATGAA | 0.1 | | chr1:−66983188 | 4 | FALSE |
| CTAAACTTATCTGACGTTGGG | ATGAG | 0.1 | | chr10:+21061613 | 5 | FALSE |
| AAAAACCAAACTAATGTTCTG | GGGGG | 0.1 | | chr9:+97484825 | 4 | FALSE |
| AAAAAGCAGTCTCACTTTGGG | AGGAG | 0.1 | | chr20:−757338 | 4 | FALSE |
| AGACAGCAATCAAACGTTGGG | AGGAG | 0.1 | | chr17:+11928614 | 4 | FALSE |
| AAAAAACCATGTAAGGTTGGG | TGGGA | 0.1 | | chr8:−94332742 | 4 | FALSE |
| CAAAATAAAACTAATGTTGGG | CTGGG | 0.1 | | chr6:+46298368 | 5 | FALSE |
| TAAAACCAATAAAAAGTTGAG | GAGAA | 0.1 | | chr1:−76468822 | 5 | FALSE |
| AAAAACCAATTTACCTTTGAG | GAGAG | 0.1 | | chrX:−103732828 | 4 | FALSE |
| TAAAACAAATACAAGGTTGGG | ACGAA | 0.1 | | chr9:+62855232 | 5 | FALSE |
| TAAAACAAATACAAGGTTGGG | ACGAA | 0.1 | | chr9:+41290897 | 5 | FALSE |
| GAAAACCAATCTCAGTTTGGA | AAGGG | 0.1 | | chr20:+43291205 | 5 | FALSE |

| MOGSB sequence | PAM | On-target Score | Off−targetScore |
|---|---|---|---|
| TATACAGGCAGTTGTTTAAGG | CAGGA | 35.8 | 84.7 |

| Sequence | PAM | Score | Gene | Chromosome | Mismatch | On-target |
|---|---|---|---|---|---|---|
| AAGACAGGCAGTTGTTTCAGA | CAGAG | 0.6 | | chr9:9250597 | 4 | FALSE |
| ATTACAGGCAGTTTTTTATGG | TAGAG | 0.5 | | chr1:+69318870 | 4 | FALSE |
| GAGACAGGCAGTTGTTTAGTG | GTGGA | 0.5 | | chr5:−170555135 | 4 | FALSE |
| TTTACAGGCAGCTGTTTTAGG | AGGGG | 0.5 | | chr7:−47773752 | 3 | FALSE |
| AATTCAGGCAGGTGTTTAATG | GAGAA | 0.5 | | chr2:−140622360 | 4 | FALSE |
| GAAAGAGGCAGTTGTTTTAGG | ATGAG | 0.5 | | chr9:+31766690 | 4 | FALSE |
| AATACATGCATTTGTTTATGG | AGGAA | 0.4 | | chrY:−5814288 | 4 | FALSE |
| GATAAAGGCAGTTGCTGAAGG | AAGAG | 0.4 | | chr2:−119219992 | 4 | FALSE |
| AATACATGCATTTGTTTATGG | AGGAA | 0.4 | | chrX:−92697525 | 4 | FALSE |
| CATACAGGAAGTTGATTAATG | AGGAA | 0.4 | | chr1:−147676835 | 4 | FALSE |
| CATTTAGGCAGTTGCTTAAGG | GTGAA | 0.4 | | chr18:+61654239 | 4 | FALSE |
| TATACAGGCAGCTGCTAAAGG | CTGGA | 0.3 | | chr11:−30396341 | 3 | FALSE |
| TATACTGACAATTGTTTAAGG | AAGAA | 0.3 | | chr3:−159702981 | 3 | FALSE |
| GATACAGGGTGTTTTTTAAGG | TGGGG | 0.3 | | chrX:−2661444 | 4 | FALSE |
| GTTACAGGCATTTGTTCAAGA | CAGAA | 0.1 | | chr16:−60644045 | 5 | FALSE |
| AGTACAGGCAGTTGATTAACC | ACGAA | 0.1 | | chr6:−116206219 | 5 | FALSE |
| CAGACAAGCAGTTGTTAAATG | TAGAG | 0.1 | | chr7:+136704248 | 5 | FALSE |
| AAAACAGGCAATTGTTTGAGA | GAGAG | 0.1 | | chr10:+26802472 | 5 | FALSE |
| GCTGCAGGCAGTTGTATGAGG | ATGAA | 0.1 | | chr7:−27126806 | 5 | FALSE |
| ATTACAGGCAATTATTTAAGA | AAGAA | 0.1 | | chr1:+90897534 | 5 | FALSE |
| AATAAATGCAGTTGTTCAAGA | TAGAA | 0.1 | | chr3:−82792950 | 5 | FALSE |
| CATGGAGGCAGATGTTTAAGA | GAGGA | 0.1 | | chr1:+5950069 | 5 | FALSE |
| AATCCGGGCAGTTGTGTAAAG | GAGAA | 0.1 | | chr21:−36833896 | 5 | FALSE |
| AATTCTGGCAGTGGTTTAAGC | AAGAG | 0.1 | | chr20:−42169459 | 5 | FALSE |
| AATATAGGCATTTGTTTAAAT | GGGAG | 0.1 | | chr22:+30120221 | 5 | FALSE |
| TAAACAGGCAATTGTTAAAAG | AGGAA | 0.1 | PRDM6 | chr5:+123192360 | 4 | FALSE |
| AAAACAGGCAGCTGTTAAAAG | GTGAA | 0.1 | | chr8:+110123016 | 5 | FALSE |
| AAGACAGGCAGTTGTGTGAGT | ACGGA | 0.1 | | chr8:−1700154 | 5 | FALSE |
| GATAGAGACAGTTGTGTAAGA | GAGGA | 0.1 | | chr3:+45454832 | 5 | FALSE |
| CAAACAGGAAGTTATTTAAAG | CAGGG | 0.1 | | chr12:+11596210 | 5 | FALSE |
| AAGAGAGGCAGTTGTATTAGG | CAGGA | 0.1 | | chr2:+152602180 | 5 | FALSE |
| AATTCAGGCAATTGTTTTAAG | ATGAA | 0.1 | | chr11:+13247842 | 5 | FALSE |
| CATTGAGGGAGTTGTTTAAGC | ATGAA | 0.1 | | chr6:−155000183 | 5 | FALSE |
| TTTAGAGGAAGTTGTTTAGGG | ATGGG | 0.1 | | chr4:+116739523 | 4 | FALSE |
| CACACAGACAGTTGCTTAGGG | CAGAA | 0.1 | | chrX:−136218777 | 5 | FALSE |
| AATAAAGGCAGTTCTTTAAAC | CTGAG | 0.1 | | chr14:−79731302 | 5 | FALSE |
| GATATAGCCAGTTTTTTAAGT | ACGAA | 0.1 | | chr5:+159188976 | 5 | FALSE |
| CATAAAGGCATTTGTTGAAGA | TAGGA | 0.1 | | chr13:+11323345 | 5 | FALSE |
| GATATAGGGAGTTATTTAAGT | CTGAG | 0.1 | | chr17:−48531927 | 5 | FALSE |
| AAGGCAGGCAGTTGGTCAAGG | CAGAA | 0.1 | | chr16:+19157660 | 5 | FALSE |
| AATATAGGCAGTTCTTTGAGC | AAGAA | 0.1 | | chr11:−14710937 | 5 | FALSE |
| CAAACACACAGTTGTTTAAAG | GAGAA | 0.1 | | chr6:+18902380 | 5 | FALSE |
| TATTCAGGCAGTTGTAAAAGA | CAGGA | 0.1 | | chr13:+81836921 | 4 | FALSE |
| TATAGAGGCAGTTGCTTATGT | AAGGA | 0.1 | | chr13:−65632940 | 4 | FALSE |
| TATGCTGGGAGTTGTTTAAAG | GAGAA | 0.1 | | chr15:−91651756 | 4 | FALSE |
| TGTTCAGGCTGTTGTTCAAGG | CTGGA | 0.1 | | chr2:−175748993 | 4 | FALSE |
| TATACTGGCTGTTGCTTAAGA | AAGAA | 0.1 | | chr3:+83732988 | 4 | FALSE |
| ATTACAGGCAGATCTTTGAGG | TGGAG | 0.1 | | chr13:+51261581 | 5 | FALSE |
| CATGGAGGCAGGTGTTTTAGG | CAGAA | 0.1 | | chr10:+12939256 | 5 | FALSE |
| TATAAAAGCAATTGTTTAATG | AGGAA | 0.1 | | chr21:+33525802 | 4 | FALSE |

TABLE 2
PCR primers for off target analysis. Table
discloses SEQ ID NOS 127-146, respectively,
in order of appearance.
OFF target analysis primers

| | | |
|---|---|---|
| MOGSA | MOGSAOTChr1F | 5′-AAGCCTTCAGGCAGCATCTT-3′ |
| OFF | | |
| targets | | |
| | MOGSAOTChr1R | 5′-ACTGGTCACCGAAGTGTTCA-3′ |
| | MOGSAOTChr18F | 5′-GCCCAACTGGATAAGGACCC-3′ |
| | MOGSAOTChr18R | 5′-GCATGTGGCATCATCAGACC-3′ |
| | MOGSAOTChr7F | 5′-CTGCTTCCCTGCCCACTTCT-3′ |
| | MOGSAOTChr7R | 5′-CGTTCTGTGGCACCATAAGC-3′ |
| | MOGSAOTChr2F | 5′-AGTACGCAGAGAGCTCCCGA-3′ |
| | MOGSAOTChr2R | 5′-CCCAGGTCCATGGAAACCAA-3′ |
| | MOGSAOTChr15F | 5′-CCTCCATCCCTCAGGAGTGC-3′ |
| | MOGSAOTChr15R | 5′-TGCGTGCCCCATTCCTAGTC-3′ |
| MOGSB | MOGSBOTChr9F | 5′-CTCGCTCACCGGGGTATCTG-3′ |
| OFF | | |
| targets | | |
| | MOGSBOTChr9R | 5′-CTGGCACAGGTGTCCAGGGT-3′ |
| | MOGSBOTChr1F | 5′-AGGGTTGACCTTGGATGAGC-3′ |
| | MOGSBOTChr1R | 5′-CCCATTAGCCCGGGATCACA-3′ |
| | MOGSBOTChr5F | 5′-TTTGAGAAGCTGGCTGGCGA-3′ |
| | MOGSBOTChr5R | 5′-CTGGCTCCTGGAGGGGTAAC-3′ |
| | MOGSBOTChr7F | 5′-CCTGCTAACCTGCGTTCTGT-3′ |
| | MOGSBOTChr7R | 5′-GGCAGGCTTTGCCTCATTTC-3′ |
| | MOGSBOTPRDM6F | 5′-GTGCAATTACTGCCATGCCT-3′ |
| | MOGSBOTPRDM6R | 5′-CCTTCCTGCCTGTTGCCATT-3′ |

What is claimed:

1. A composition for preventing or treating a retroviral infection in vitro or in vivo, the composition comprising:
i. a first isolated nucleic acid sequence encoding a first Clustered Regularly Interspaced Short Palindromic Repeat (CRISPR)-associated endonuclease and at least one guide RNA (gRNA), the gRNA being complementary to a target sequence in the integrated retroviral DNA;
ii. a second isolated nucleic acid sequence encoding a second Clustered Regularly Interspaced Short Palindromic Repeat (CRISPR)-associated endonuclease and at least one guide RNA (gRNA), the gRNA being complementary to a target sequence in the integrated retroviral DNA;
iii. a third isolated nucleic acid sequences encoding a third Clustered Regularly Interspaced Short Palindromic Repeat (CRISPR)-associated endonuclease and at least one guide RNA (gRNA), the gRNA being complementary to a target sequence in a mannosyl-oligosaccharide glucosidase (MOGS) gene;
iv. a fourth isolated nucleic acid sequences encoding a fourth Clustered Regularly Interspaced Short Palindromic Repeat (CRISPR)-associated endonuclease and at least one guide RNA (gRNA), the gRNA being complementary to a target sequence in a mannosyl-oligosaccharide glucosidase (MOGS) gene regulatory region.

2. The composition of claim 1, wherein the first and second target sequences comprise one or more nucleic acid sequences in the retrovirus, the target sequences comprising:
long terminal repeat (LTR) nucleic acid sequences, Gag nucleic acid sequences, nucleic acid sequences encoding structural proteins, non-structural proteins or combinations thereof.

3. The composition of claim 1, wherein intervening sequences between the first and second target sequences are excised.

4. The composition of claim 1, wherein the target sequences comprise one or more intron sequences, exon sequences or combinations thereof of the MOGS gene.

5. The composition of claim 4, wherein the regulatory target sequences of the MOGS gene comprise a promoter sequence, an enhancer, coding or non-coding regions associated with the MOGS gene.

6. The composition of claim 4, wherein intervening sequences between the first and second target sequences are excised.

7. The composition of claim 1, wherein the retrovirus is a human immunodeficiency virus (HIV).

8. A composition for preventing or treating a human immunodeficiency virus (HIV) infection, the composition comprising at least two isolated nucleic acid sequences wherein:
(i) a first isolated nucleic acid sequence encodes a first Clustered Regularly Interspaced Short Palindromic Repeat (CRISPR)-associated endonuclease and at least one guide RNA (gRNA), the gRNA being complementary to a target sequence in encoding a third Clustered Regularly Interspaced Short Palindromic Repeat (CRISPR)-associated endonuclease and at least one guide RNA (gRNA), the gRNA being complementary to a target sequence in a mannosyl-oligosaccharide glucosidase (MOGS) gene;
(ii) a second isolated nucleic acid sequences encoding a fourth Clustered Regularly Interspaced Short Palindromic Repeat (CRISPR)-associated endonuclease and at least one guide RNA (gRNA), the gRNA being complementary to a target sequence in a mannosyl-oligosaccharide glucosidase (MOGS) gene.

9. The composition of claim 8, wherein the target sequences of the MOGS gene comprise promoter sequences, enhancer sequences, intron sequence, exon sequences and combinations thereof.

10. The composition of claim 8, further comprising two or more isolated nucleic acid sequences encoding two or more (CRISPR)-associated endonucleases and guide RNAs (gRNAs) having complementarity to two or more target sequences, the target sequences comprising HIV sequences, sequences in receptors used by HIV for attachment and/or infection of a cell, and combinations thereof.

11. The composition of claim 10, wherein the at least one receptor comprises CCR5, CD4, variants or combinations thereof.

12. The composition of claim 10, wherein the HIV target sequences comprise one or more nucleic acid sequences comprising: long terminal repeat (LTR) nucleic acid sequences, Gag nucleic acid sequences, nucleic acid sequences encoding structural proteins, non-structural proteins or combinations thereof.

13. The composition of claim 8, further comprising a therapeutically effective amount of at least one antiretroviral agent.

14. A method of preventing and treating infection by a retrovirus in vitro or in vivo, comprising administering at least two isolated nucleic acid sequences wherein:
iii) a first isolated nucleic acid sequence encodes a first Clustered Regularly Interspaced Short Palindromic Repeat (CRISPR)-associated endonuclease and at least one guide RNA (gRNA), the gRNA being complementary to a target sequence in encoding a third Clustered Regularly Interspaced Short Palindromic Repeat (CRISPR)-associated endonuclease and at least one guide RNA (gRNA), the gRNA being complementary to a target sequence in a mannosyl-oligosaccharide glucosidase (MOGS) gene;
(iv) a second isolated nucleic acid sequences encoding a second Clustered Regularly Interspaced Short Palindromic Repeat (CRISPR)-associated endonuclease and at least one guide RNA (gRNA), the gRNA being complementary to a target sequence in a mannosyl-oligosaccharide glucosidase (MOGS) gene;
wherein intervening sequences between the first and second target sequences are excised.

15. The method of claim 14, wherein the target sequences of the MOGS gene comprise promoter sequences, enhancer sequences, intron sequences, exon sequences and combinations thereof.

16. The method of claim 14, further comprising administering two or more isolated nucleic acid sequences encoding two or more (CRISPR)-associated endonucleases and guide RNAs (gRNAs) having complementarity to two or more target sequences, the target sequences comprising HIV sequences, sequences in receptors used by HIV for attachment and/or infection of a cell, and combinations thereof.

17. The method of claim 16, wherein the at least one receptor comprises CCR5, CD4, variants or combinations thereof.

18. The method of claim 16, wherein the HIV target sequences comprise one or more nucleic acid sequences comprising: long terminal repeat (LTR) nucleic acid sequences, Gag nucleic acid sequences, nucleic acid sequences encoding structural proteins, non-structural proteins or combinations thereof.

19. The method of claim 14, further comprising administering a therapeutically effective amount of at least one antiretroviral agent.

20. A composition for preventing or treating a human immunodeficiency virus (HIV) infection, the composition comprising an isolated nucleic acid sequence encoding a Clustered Regularly Interspaced Short Palindromic Repeat (CRISPR)-associated endonuclease; and at least one guide RNA (gRNA), a first gRNA being complementary to a first target nucleic acid sequence within a mannosyl-oligosaccharide glucosidase (MOGS) gene and a second gRNA being complementary to a second target nucleic acid sequence within the MOGS gene that is different from the first target nucleic acid sequence, wherein the CRISPR-associated endonuclease, the first gRNA, and the second gRNA are configured to excise the region between the first target nucleic acid sequence and the second target nucleic acid sequence.

21. The composition of claim 20, wherein the target sequences of the MOGS gene comprise promoter sequences, enhancer sequences, intron sequences, exon sequences and combinations thereof.

22. A vector encoding for the isolated nucleic acids of claim 1.

23. A vector encoding for the isolated nucleic acids of claim 8.

24. A vector encoding for the isolated nucleic acids of claim 20.
